(19) United States
(12) Patent Application Publication
(10) Pub. No.: US 2023/0180778 A1
(43) Pub. Date: Jun. 15, 2023

(54) MILK FERMENTATION PROCESS

(71) Applicant: Fatah Bashir Ahtesh, North Werribee (AU)

(72) Inventor: Fatah Bashir Ahtesh, North Werribee (AU)

(21) Appl. No.: 17/916,244

(22) PCT Filed: Apr. 1, 2021
(86) PCT No.: PCT/AU2021/050309

§ 371 Date: Sep. 30, 2022

(30) Foreign Application Priority Data
Apr. 2, 2020 (AU) 2020901020

(51) Int. Cl. A23C 9/123; A23C 9/12; A23C 9/127; A23J 3/08

(52) U.S. Cl. CPC A23C 9/1234; A23C 9/1203; A23C 9/1275; A23J 3/08

(57) ABSTRACT

The present disclosure relates to processes for producing antihypertensive peptide compositions from milk compositions. It also relates to peptide compositions producible by the processes, as well as dosage forms, food products, supplements, powdered milk formula and beverages containing the peptide compositions, and to therapeutic methods and uses of the peptide compositions.

### FIELD

The present disclosure relates to processes for producing antihypertensive peptide compositions from milk compositions. It also relates to peptide compositions producible by the processes, as well as dosage forms, food products, supplements, powdered milk formula and beverages containing the peptide compositions, and to therapeutic methods and uses of the peptide compositions.

### BACKGROUND

The discussion of the background to the disclosure is intended to facilitate an understanding of the disclosure. However, it should be appreciated that the discussion is not an acknowledgement or admission that any of the material referred to was published, known or part of the common general knowledge as at the priority date of the application.

Developing and growing agricultural products, food industrialisation, and mechanisation have led to dramatic changes in lifestyle, particularly dietary pattern, which in turn have produced increased occurrence in chronic diseases such as cardiovascular disease, stroke, diabetes, hypertension and cancer. Indeed, obesity, hypertension and cardiovascular diseases have increased at an alarming rate worldwide in the last two decades, nearly 2.5-fold in Australia compared to the USA (Cameron et al., 2003; Roberfroid, 1999, 2000; World Health Organisation, 2002, 2003). Consequently, in recent years, people seeking healthier lifestyle prefer diets with low or fat free foods; this has led to the development of functional foods (Roberfroid, 1999a; Food and Agriculture Organization of the United Nations, & World Health Organization, 2002; Cameron et al., 2003; World Health Organization, 2003).

Functional foods are defined as foods ‘that can beneficially affect one or more target functions in the body, beyond the adequate nutritional effect, in a way relevant to improved state of health and well-being and/or reduce the risk of disease’ (Contor, 2001). Milk products, particularly fermented milk containing probiotics are most popular in this category of foods (Stanton et al., 2005). Probiotics are defined as ‘live microorganisms which, when consumed in adequate amounts, confer a health benefit on the host’ (World Health Organisation, 2002). The benefits of utilising these organisms include maintenance of gut health, reduced allerginicity, increased bio-accessibility of lipids and proteins in foods, and lowering of blood pressure due to polyamines and bioactive peptides (Marteau et al., 1990; Santos, San Mauro, & Diaz, 2006; Tuohy et al., 2003). These bioactive peptides have the ability to reduce the risk of colorectal cancer, stimulate the immune response and reduce the risk of cancer, non-insulin dependent diabetes, obesity, cardiovascular disease and hypertension (Shah, 2007; Tuohy et al., 2003; Williams & Jackson, 2002).

However, the health conscious consumer now requires additional health benefits from these products, which have opened new areas for research (Shah, 2007). Among these, peptides with blood pressure-lowering effects have received considerable significance in being associated with the role of diet in prevention and treatment of disease (López-Fandiño, Otte, & van Camp, 2006). Blood pressure regulation is partially dependent on the rennin angiotensin system (Silva & Malcata, 2005), in which the angiotensin-I converting enzyme (ACE) regulates the peripheral blood pressure and its inhibition can exert an anti-hypertensive effect (Gobbetti, Minervini, & Rizzello, 2004).

Bioactive peptides are defined as specific protein fragments that have positive impact on body functions or conditions and may ultimately influence health (Kitts & Weiler, 2003). Upon oral administration, bioactive peptides may affect the major body systems, namely, the cardiovascular, digestive, immune and nervous systems, depending on their amino acid sequence (Erdmann, Cheung, & Schroder, 2008; FitzGerald et al., 2011; Yamamoto et al., 2010). These peptides are released through enzymatic breakdown of dairy proteins by digestive enzymes in the gastrointestinal tract (GIT) or extracellular proteinases formed by lactobacilli during their growth in milk (Seppo et al., 2003; van der Burg-Koorevaar & Schalk, 2010). The tri-peptides (a.k.a. lactotripeptides) Valyl-Prolyl-Proline (Val-Pro-Pro) and Isoleucyl-Prolyl-Proline (Ile-Pro- Pro) have been identified as antihypertensive agents, which inhibit the action of ACE (van der Burg-Koorevaar & Schalk, 2010).

Most of the probiotic microorganisms are sensitive to food acidity and oxygen availability. Short shelf-life fermented dairy products like yoghurt, are the most common functional foods on the market (Hekmat, Soltani, & Reid, 2009; Ozer et al., 2007; Stanton et al., 2003). During the fermentation process, probiotics produce a range of secondary metabolites, some of which have been associated with health promoting properties of which the notable ones are the B vitamins and bioactive peptides. The physiologically active peptides are produced from many food proteins during gastro-intestinal digestion and fermentation of food by lactic acid bacteria (LAB).

Production of ACE-I peptides in situ in dairy products is one approach for generating these peptides. One way involves fermentation with highly proteolytic strains of LAB (Gobbetti et al., 2004), a challenge to this approach, however, lies in the selection of the appropriate microorganisms, strains thereof, or a combination thereof (Gobbetti et al., 2004; Meisel, 1998).

There remains a need for further and/or improved processes for producing anti-hypertensive peptide compositions, for example compositions with high anti-hypertensive activity.

### SUMMARY

In a first aspect, there is provided a process for producing an antihypertensive peptide composition, comprising:
a) fermenting a milk composition with a Lactobacillus helveticus and a peptidase selected from the group consisting of an aminopeptidase and a serine endopeptidase, thereby at least partially hydrolysing peptides present in the composition; and
b) heat-treating the product of step a) so as to inactivate the Lactobacillus helveticus and the peptidase;
c) admixing the at least partially hydrolysed peptides with a further quantity of milk composition and with Kluyveromyces marxianus, and fermenting the mixture to produce the antihypertensive peptide composition.

In some embodiments, the milk composition is reconstituted skimmed milk. In some embodiments, the milk composition is pasteurised. In some embodiments, the milk composition is pasteurised by heating it at a temperature in the range of from 85° C. to 95° C. for a period in the range of from 15 minutes to 18 minutes.

In some embodiments, prior to step a), the milk composition is subjected to mechanical agitation. In some embodiments, mechanical agitation is carried by agitating the milk composition at a rate in the range of from 200 to 225 revolutions per minute, at a temperature of about 37° C., and for a period in the range of from 30 minutes to 1 hour.

In some embodiments, prior to step a), the Lactobacillus helveticus is activated by carrying out three iterations of incubating in MRS medium at about 37° C. for about 18 hours.

In some embodiments, the peptidase is an aminopeptidase. In some embodiments, the aminopeptidase is present in a composition having an activity of about 1000 leucine amino-peptidase (LAPU g⁻¹).

In some embodiments, the peptidase is a serine endopeptidase. In some embodiments, the serine endopeptidase is present in a composition having an activity of at least 2.4 Anson units per g.

In some embodiments, to commence fermentation, an amount in the range of from 1 to 2% v/v of a composition comprising the Lactobacillus helveticus, and an amount in the range of from 0.04 to 0.45% w/w of a peptidase composition is added to the milk composition.

In some embodiments, the first fermentation step is carried out for a period in the range of from 8 to 12 hours.

In some embodiments, in step b) the composition is subjected to a temperature in the range of from 80° C. to 90° C. for a period in the range of from 12 to 15 minutes.

In some embodiments, the further quantity of milk composition is pasteurised reconstituted skimmed milk.

In some embodiments, the second fermentation step is carried out for a period in the range of from 8 to 12 hours.

In some embodiments, following fermentation of the mixture containing Kluyveromyces marxianus, the composition is subjected to heat treatment to inactivate the Kluyveromyces marxianus. In some embodiments, following fermentation of the mixture containing Kluyveromyces marxianus, the composition is subjected to a temperature in the range of from 80° C. to 90° C. for a period in the range of from 6 to 30 minutes.

In some embodiments, following heat treatment, antihypertensive peptide is separated from Kluyveromyces marxianus. In some embodiments, following separation from Kluyveromyces marxianus, the antihypertensive peptide composition is lyophilised.

In a second aspect, there is provided a process for producing an antihypertensive peptide composition, comprising:
i) subjecting a milk composition to mechanical agitation;
ii) fermenting the product of step i) with a Lactobacillus helveticus and a peptidase selected from the group consisting of an aminopeptidase and a serine endopeptidase, thereby at least partially hydrolysing peptides present in the composition; and
iii) heat-treating the product of step ii) so as to inactivate the Lactobacillus helveticus and the peptidase.

In some embodiments, the milk composition is reconstituted skimmed milk. In some embodiments, the milk composition is pasteurised. In some embodiments, the milk composition is pasteurised by heating it at a temperature in the range of from 85° C. to 95° C. for a period in the range of from 15 minutes to 18 minutes.

In some embodiments, in step i) mechanical agitation is carried by agitating the milk composition at a rate in the range of from 200 to 225 revolutions per minute, at a temperature of about 37° C., and for a period in the range of from 30 minutes to 1 hour.

In some embodiments, prior to step ii), the Lactobacillus helveticus is activated by carrying out three iterations of incubating in MRS medium at about 37° C. for about 18 hours.

In some embodiments, the peptidase is an aminopeptidase. In some embodiments, the aminopeptidase is present in a composition having an activity of about 1000 leucine amino-peptidase (LAPU g⁻¹).

In some embodiments, the peptidase is a serine endopeptidase. In some embodiments, the serine endopeptidase is present in a composition having an activity of at least 2.4 Anson units per g.

In some embodiments, to commence fermentation, an amount in the range of from 1 to 2% v/v of a composition comprising the Lactobacillus helveticus, and an amount in the range of from 0.04 to 0.45% w/w of a peptidase composition is added to the milk composition.

In some embodiments, step ii) is carried out for a period in the range of from 8 to 12 hours.

In some embodiments, in step iii) the composition is subjected to a temperature in the range of from 80° C. to 90° C. for a period in the range of from 12 to 15 minutes.

In some embodiments, following step iii), antihypertensive peptides are separated from Lactobacillus helveticus. In some embodiments, following separation from Lactobacillus helveticus, the antihypertensive peptide composition is lyophilised.

In some embodiments, following step iii) the antihypertensive peptide composition is admixed with a further quantity of milk composition and with Kluyveromyces marxianus, and the resulting mixture is fermented to produce a further antihypertensive peptide composition. In some embodiments, the further quantity of milk composition is pasteurised reconstituted skimmed milk. In some embodiments, the pasteurised reconstituted skimmed milk has been subjected to mechanical agitation. In some embodiments, the mixture containing Kluyveromyces marxianus is fermented for a period in the range of from 8 to 12 hours. In some embodiments, following fermentation of the mixture containing Kluyveromyces marxianus, the composition is subjected to heat treatment at a temperature in the range of from 80° C. to 90° C. for a period in the range of from 6 to 30 minutes, so as to inactivate the Kluyveromyces marxianus. In some embodiments, following heat treatment, antihypertensive peptide is separated from Kluyveromyces marxianus. In some embodiments, following separation from Kluyveromyces marxianus, the antihypertensive peptide composition is lyophilised.

In some embodiments of either the first or second aspects, the peptide composition has antioxidant activity, antibacterial activity, anticancer activity, antihypertensive activity and/or facilitates weight loss.

In a third aspect, there is provided an antihypertensive peptide composition produced according to a process of the first or second aspects.

In a fourth aspect, there is provided a dosage form comprising the antihypertensive peptide composition of the third aspect.

In some embodiments, the dosage form is in the form of a liquid, powder, tablet or capsule.

In a fifth aspect, there is provided a food product or supplement comprising the antihypertensive peptide composition of third aspect.

In some embodiments, the food product is a spread or a yogurt.

In a sixth aspect, there is provided a powdered milk formula comprising the antihypertensive peptide composition of the third aspect.

In a seventh aspect, there is provided a beverage comprising the antihypertensive peptide composition of the third aspect.

In some embodiments, the beverage is a milk drink.

In some embodiments of the dosage form of the fourth aspect, the food product or supplement of the fifth aspect, the powdered milk formula of the sixth aspect or the beverage of the seventh aspect, the dosage form, food product or supplement, powdered milk formula or beverage comprises one or more of a flavouring, antioxidant and sweetener.

In an eighth aspect, there is provided a method of treating, preventing, or reducing the likelihood of a disease or disorder in a subject, comprising administering an effective amount of an antihypertensive peptide composition, dosage form, food product, food supplement, powdered milk formula or beverage according to the third to seventh aspects to the subject.

In some embodiments, the disease or disorder is hypertension, a cardiovascular disease or disorder, heart disease, an immune system disease or disorder, a bacterial infection, a viral infection, cancer, being overweight, or obesity. In some embodiments, the disease or disorder is selected from the group consisting of hypertension and heart disease. In some embodiments, the disease or disorder is being overweight or obesity.

In a ninth aspect, there is provided a use of an antihypertensive peptide composition, dosage form, food product, food supplement, powdered milk formula or beverage according to any of the third to seventh aspects, for the manufacture of a medicament for the treatment, prevention, or for reducing the likelihood of a disease or disorder selected from the group consisting of hypertension, a cardiovascular disease or disorder, heart disease, an immune system disease or disorder, a bacterial infection, a viral infection, cancer, being overweight, and obesity.

In a tenth aspect, there is provided an antihypertensive peptide composition, dosage form, food product, powdered milk formula or beverage according to any of the third to seventh aspects, for use in the treatment or prevention of, or for use in reducing the likelihood of, a disease or disorder.

In some embodiments, the disease or disorder is selected from the group consisting of hypertension, a cardiovascular disease or disorder, heart disease, an immune system disease or disorder, a bacterial infection, a viral infection, cancer, being overweight, and obesity.

### BRIEF DESCRIPTION OF THE DRAWINGS

FIG. 1 is a flowchart setting out an embodiment of the process.

FIG. 2 is a graph showing a change of body weight of spontaneously hypertensive rats orally administered with the fermented peptide composition (FC), control 1 (NFC), or control 2 (NC). All data expressed as mean ±SEM (n=9).

FIG. 3 is a graph showing the amount of feed intake of spontaneously hypertensive rats orally administered with the fermented peptide composition (FC), control 1 (NFC), or control 2 (NC). All data expressed as mean ±SEM (n=9).

### DETAILED DESCRIPTION

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or groups of compositions of matter. Thus, as used herein, the singular forms “a”, “an” and “the” include plural aspects unless the context clearly dictates otherwise. For example, reference to “a” includes a single as well as two or more; reference to “an” includes a single as well as two or more; reference to “the” includes a single as well as two or more and so forth.

Each embodiment of the present disclosure described herein is to be applied mutatis mutandis to each and every other embodiment unless specifically stated otherwise. The present disclosure is not to be limited in scope by the specific examples or embodiments described herein, which are intended for the purpose of exemplification only. Functionally-equivalent products, compositions and methods are clearly within the scope of the disclosure as described herein.

The term “and/or”, e.g., “X and/or Y” shall be understood to mean either “X and Y” or “X or Y” and shall be taken to provide explicit support for both meanings or for either meaning.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the present specification, including definitions, will control. In addition, the materials, methods, and examples are illustrative only and not intended to be limiting.

The term “about” as used herein means within 5%, and more preferably within 1%, of a given value or range. For example, “about 3.7%” means from 3.5 to 3.9%, preferably from 3.66 to 3.74%. When the term “about” is associated with a range of values, e.g., “about X% to Y%”, the term “about” is intended to modify both the lower (X) and upper (Y) values of the recited range. For example, “about 20% to 40%” is equivalent to “about 20% to about 40%”.

The term “pharmaceutically acceptable” with respect to a substance as used herein means that substance which is suitable for use in contact with the tissues of humans and lower animals without undue toxicity, irritation, allergic response, and the like, commensurate with a reasonable benefit/risk ratio, and effective for the intended use when the substance is used in a pharmaceutical composition

The term “therapeutically effective amount” as used herein refers to an amount of active ingredient needed to provide a desired level of active ingredient in the bloodstream or at a target organ of to provide an anticipated physiological response. The precise amount will vary in response to several factors including, but not limited to, the type of active ingredient, bioavailability of the active ingredient, patient characteristics (e.g. age, weight, gender), severity of symptoms, contraindications, and so forth. A therapeutically effective amount of an active ingredient may be administered in a single dosage, or through multiple dosages of an amount that cumulatively provides a therapeutic effect. The ‘therapeutic effect’ may reduce the severity of a disease, medical condition or one or more associated symptoms, and/or may be therapeutic in terms of a partial or complete cure of a disease or medical condition.

### Processes for Producing Antihypertensive Peptide Compositions

The first aspect provides a process for producing an antihypertensive peptide composition. The process provides fermented peptidic compositions having unexpectedly high ACE inhibitory activity.

Step a) of the process involves fermenting a milk composition with a Lactobacillus helveticus and a peptidase.

Any suitable milk composition may be used. In some embodiments, the milk composition is milk, for example semi-skimmed or skimmed milk. For example, it may be a milk composition having 3-5 g/100 g protein, 0.05-0.2 g/100g fat, and 4-7 g/100 g sugars, or it may be a milk composition having 3.5 g/100 g protein, 0.1 g/100 g fat, and 5.3 g/100 g sugars. In some embodiments, the milk composition is a whey protein isolate or concentrate. In some embodiments, the milk composition is a reconstituted milk composition, such as that produced by admixing appropriate quantities of milk powder with water, e.g. distilled water. In some embodiments, the milk composition is a reconstituted semi-skimmed milk. In some embodiments, the milk composition is reconstituted skimmed milk (RSM). For example, it may be reconstituted skimmed milk produced from a powder composition containing 40-70% sugars (e.g. lactose), 30-50% protein, and 0.5-2% fat, or from a powder composition containing 52% sugars (e.g. lactose), 37% protein and 1.2% fat. In some embodiments, the milk composition is reconstituted skimmed milk having from 8% to 16% w/v skim milk powder, for example about 12% w/v skim milk powder, admixed with distilled water. In some embodiments, the milk composition is reconstituted whey protein concentrate, e.g. having from 2 to 6% w/v whey protein concentrate, for example 4% w/v whey protein concentrate, admixed with distilled water.

The milk composition has typically been treated prior to use to at least partially sterilise the composition. In some embodiments, the milk composition is a pasteurised milk composition, for example a pasteurised reconstituted skimmed milk composition. Pasteurisation is a technique used for partial sterilization of a product such as milk, involving heating for a suitable period of time, typically at a temperature of less than 100° C.

Pasteurisation may be carried out as part of the process, for example as a step of the process. Alternatively, pasteurisation of the milk composition may be carried out separately from carrying out the main process. For example pasteurisation of a milk composition may be carried out at a farm or a dairy plant, prior to shipping of the pasteurised milk composition to the site where the process is carried out.

In some embodiments, the milk composition is pasteurised by heating it at a temperature in the range of from 85° C. to 95° C., for example at a temperature of about 90° C. In some embodiments, prior to mechanical hydrolysis, the milk composition is pasteurised by heating it for a period in the range of from 15 seconds to 1 hour, for example for a period in the range of from 1 minute to 30 minutes, from 5 minutes to 30 minutes, from 10 minutes to 30 minutes, from 20 minutes to 30 minutes, from 5 minutes to 20 minutes, from 10 minutes to 20 minutes, from 15 minutes to 20 minutes, from 15 minutes to 18 minutes, or about 15 minutes.

In some embodiments, the milk composition is pasteurised by heating it at a temperature in the range of from 85° C. to 95° C. for a period in the range of from 15 minutes to 18 minutes, for example by heating it at about 90° C. for a period of about 15 minutes.

Prior to carrying out step a), in some embodiments, the process involves subjecting the milk composition to mechanical agitation. It is believed that the use of mechanical agitation results in partial hydrolysis of peptides, and facilitates the production of improved hydrolysed peptidic compositions having particularly high levels of anti-hypertensive activity. Any suitable means of agitating the milk composition may be employed. For example, shaking of the vessel containing the pasteurised milk composition may be used. Alternatively, a stirrer or impeller may be used. An agitation rate of from 100 rpm to 600 rpm may be used, for example. In some embodiments, the milk composition is agitated at a rate in the range of from 100 rpm to 600 rpm, or from 150 rpm to 400 rpm, or from 200 rpm to 300 rpm, or from 200 rpm to 225 rpm. The mechanical agitation step may for example be carried out for a time period in the range of from 15 minutes to 3 hours, from 30 minutes to 2 hours, or from 30 minutes to 1 hour. The mechanical agitation step can be conducted at any suitable temperature. In some embodiments, the mechanical hydrolysis step is carried out at about 37° C.

In some embodiments, mechanical agitation is carried out by agitating the milk composition at a temperature of about 37° C. for a period in the range of from 30 minutes to 1 hour. In some embodiments, mechanical agitation is carried by agitating the milk composition at a rate in the range of from 200 to 225 revolutions per minute, at a temperature of about 37° C., and for a period in the range of from 30 minutes to 1 hour.

Step a) involves fermenting the milk composition with a Lactobacillus helveticus and a peptidase, thereby at least partially hydrolysing peptides present in the composition, e.g. to produce a composition containing high levels of tripeptides such as Val-Pro-Pro and Ile-Pro-Pro.

Lactobacillus helveticus is a type of lactic acid bacteria which can be used in the fermentation of milk for the manufacture of some cheeses. Any suitable strain of Lactobacillus helveticus may be utilised in the present process. However, in some preferred embodiments, the Lactobacillus helveticus is Lactobacillus helveticus 881315, Lactobacillus helveticus 881188, or a mixture of the two. Fermentation of the milk composition using those strains of bacteria has been found to result in production of fermented peptide composition having high antihypertensive properties. Lactobacillus helveticus 881315 and 881188 may be obtained from commercial sources, for example from Dairy Innovation Australia Ltd (now Chr. Hansen Ltd.) Werribee, VIC, Australia.

Prior to the fermentation step, the Lactobacillus helveticus strain or strains is/are activated, for example by transferring an aliquot of the strain into a suitable broth (e.g. Man, Rogosa, and Sharpe (MRS) broth) and incubating, e.g. at 37° C. and for a period in the range of from 12 hours to 24 hours. Any suitable broth or media may be used. In some embodiments, a dairy media is used. In some embodiments, MRS broth is used. MRS broth may be obtained for example from Sigma Aldrich. In some embodiments, multiple iterations of subculturing the Lactobacillus helveticus are carried out in order to activate the bacteria, prior to using the strain to ferment the milk composition. In some embodiments, prior to step a), the Lactobacillus helveticus is activated by three iterations of incubating in MRS medium at about 37° C. for 12-18 hours, and replacing at least a portion of the MRS medium. In some embodiments, the media in which the bacteria is activated is exchanged for a milk composition (e.g. pasteurised reconstituted skimmed milk) prior to adding the Lactobacillus helveticus to the milk composition to be fermented and carrying out step a).

Typically, the cell count of the composition containing the activated bacteria, which is added to the milk composition, is in the range of from 10⁷ to 10¹¹ CFU (colony forming units)/mL. Following addition to the milk composition, the Lactobacillus helveticus will typically be present at a cell count in the range of from 10⁵ to 10⁹ CFU/mL. In some embodiments, prior to addition to the milk composition, the concentration of bacteria in the activated culture is in the range of from 10⁸ to 10¹¹ CFU/mL, or in the range of from 10⁸ to 10⁹ CFU/mL. In some embodiments, the concentration of bacteria in the activated culture is about 10¹¹ CFU/mL, or about 10^{10.8} CFU/mL.

A peptidase selected from the group consisting of an aminopeptidase and a serine endopeptidase is also used.

In some embodiments, the peptidase is an aminopeptidase. Aminopeptidases are enzymes which catalyse cleavage of amino acids from the amino terminus (N-terminus) of proteins and polypeptides. In some embodiments, the aminopeptidase is a leucine aminopeptidase (i.e. an aminopeptidase which preferentially cleaves leucine residues). In some embodiments, the aminopeptidase preferentially cleaves the N-terminal amino acid from a sequence Xaa-Yaa, in which Xaa is preferably Leu or Pro, and Yaa is Pro.

In some embodiments the aminopeptidase enzyme is a fungal protease/peptidase complex produced by submerged fermentation of a strain of Aspergillus oryzae which contains both endoprotease and exopeptidase activities. In some embodiments, the aminopeptidase is present in the form of a composition having an activity in the range of from 800-1200 leucine amino-peptidase (LAPU g⁻¹), or about 1000 leucine amino-peptidase (LAPU g⁻¹), for example such as the Flavourzyme® 1000 L product (EC 3.4.11.1) available from Novozymes Australia, NSW, Australia.

In some embodiments, the peptidase is a serine endopeptidase, for example a subtilisin. For example a serine endopeptidase obtained from Bacillus licheniformis may be used, and/or for example present in the form of a composition having an activity of at least 1 Anson units per gram, or at least 2 Anson units per gram, or at least 2.4 Anson units per gram, or in the range of from 1 to 5 Anson units per gram. One Anson unit is defined as the amount of enzyme which digests urea-denatured hemoglobin such that there is liberated an amount pf TCA-soluble product per minute which gives the same colour with Folin-Ciocalteu Phenol reagent as one milliequivalent of tyrosine at 25° C. at pH 7.50. One such example is the product sold under the trade name Alcalase®, available from Sigma-Aldrich.

A suitable amount of Lactobacillus helveticus and a suitable amount of peptidase are added to the milk composition.

For example, in some embodiments, an amount in the range of from 0.5 to 5% v/v of a composition comprising the Lactobacillus helveticus is added, or an amount in the range of from 0.5% to 3% v/v, or an amount in the range of from 1% to 2% v/v, or an amount which is about 1% v/v.

In some embodiments, an amount in the range of from 0.02 to 1% w/w of a peptidase composition is added to the milk composition, or an amount in the range of from 0.04 to 0.45% w/w is added, or an amount of about 0.14 w/w%.

In some embodiments, an amount of an aminopeptidase composition having an activity in the range of from 800-1200 LAPU g⁻¹, and which is in the range of from 0.02 to 1% w/w of the milk composition is added.

In some embodiments, an amount of an aminopeptidase composition having an activity in the range of from 800-1200 LAPU g⁻¹, and which is in the range of from 0.04 to 0.45% w/w of the milk composition is added, e.g. about 0.14 w/w%.

In some embodiments, to commence fermentation, an amount in the range of from 1 to 2% v/v of a composition comprising the Lactobacillus helveticus, and an amount in the range of from 0.04 to 0.45% w/w of a peptidase composition is added to the milk composition.

In some embodiments, to commence fermentation, an amount in the range of from 1 to 2% v/v of a composition comprising the Lactobacillus helveticus present at a concentration in the range of from 10⁸ to 10⁹ CFU/mL, and an amount in the range of from 0.04 to 0.45% w/w of an aminopeptidase composition having an activity in the range of from 800-1200 LAPU g⁻¹, is added to the milk composition.

The first fermentation step is carried out for a period suitable to effect high levels of hydrolysis of the peptides in the milk composition. For example, in some embodiments the first fermentation step is carried out for a period in the range of from 4 to 36 hours, or 4 to 24 hours, or from 8 to 12 hours, or about 12 hours. Typically, the fermentation step is carried out at a temperature of about 37° C., although any suitable temperature may be used. Preferably, the temperature is maintained at a constant level during the fermentation step.

Typically, the first fermentation step generates quantities of lactic acid, such that the pH of the mixture decreases over the course of the fermentation step. Preferably, the initial pH of the fermentation step is in the range of from 6 to 7, for example 6.3 to 6.7, or about 6.5. By the end of the fermentation, the final pH may have decreased for example to a pH in the range of from 3 to 4, for example in the range of from 3.3 to 3.5, or at about pH 3.4.

The first fermentation step may be carried out in a suitable bioreactor, bio-fermentation apparatus, or using an incubator. The bioreactor may for example contain a thermal jacket, an agitation system (e.g. including an impeller or other means for stirring/agitating the mixture), and may contain sensors/probes for monitoring conditions such as temperature and pH. An example of a suitable bioreactor is the Biostat® A plus bioreactor. Typically, the mixture is agitated/stirred during the fermentation step. In some embodiments, the fermentation step is carried out with agitation at a rate in the range of from 100 to 500 revolutions per minute, or from 150 to 300 revolutions per minute, or from 200 to 225 revolutions per minute.

Step b) of the process involves heat-treating the product of step a). The heat treatment is sufficient to inactivate the Lactobacillus helveticus and the peptidase. The heat treatment also substantially avoiding denaturing or otherwise adversely affecting the quality of the hydrolysed milk peptides. Typically, the heat treatment step involves subjecting the product of step a) to a temperature in the range of from 80 to 90° C. for a suitable period of time, e.g. in the range of from 10 minutes to 25 minutes. In some embodiments, in step b) the composition is subjected to a temperature in the range of from 80° C. to 90° C. for a period in the range of from 12 to 15 minutes. In some embodiments, in step b) the composition is subjected to a temperature of about 85° C. for a period in the range of from 12 to 15 minutes.

Once the heat-treatment step is complete, the peptidic composition may for example be cooled to room temperature, and may be subjected to further processing.

In some embodiments, following step b), hydrolysed peptide is separated from other components of the mixture. For example, in some embodiments, following step b), hydrolysed peptide is separated from Lactobacillus helveticus. In some embodiments, following step b), hydrolysed peptide is separated from peptidase. Any suitable separation technique may be used, for example such as centrifugation. However, for some applications, it may not be necessary to separate the hydrolysed peptide from other components of the mixture. Thus, in some embodiments, following step b) the peptide composition may be used without separation or purification.

In step c), the at least partially hydrolysed peptides are admixed with a further quantity of milk composition and with Kluyveromyces marxianus, and the mixture is fermented to produce the antihypertensive peptide composition. It has also been found that, by carrying out further fermentation of the at least partially hydrolysed peptide composition, compositions having further improved ACE inhibitory activity can be obtained. It is believed that step c) results in the production of a composition having particularly high levels of tripeptides such as Val-Pro-Pro and Ile-Pro-Pro. It is also considered that carrying out a second fermentation step with Kluyveromyces marxianus separately from the first fermentation step with Lactobacillus helveticus, facilitates the production of such compositions having high levels of ACE-inhibitory activity

Kluyveromyces marxianus is a yeast having fast growth rate and thermotolerance. Any suitable Kluyveromyces marxianus may be used in the process. In some embodiments, the Kluyveromyces marxianus is Kluyveromyces marxianus LAF4. Kluveromyces marxianus LAF4 may for example be obtained from Dairy Innovation Australia Ltd (now Chr. Hansen Ltd.) Werribee, VIC, Australia.

Prior to the second fermentation step, the Kluyveromyces marxianus may be activated, for example by transferring an aliquot of the strain into a suitable medium, e.g. sterile MYGP medium (a medium containing malt extract, yeast extract, glucose, peptone) and incubating, e.g. at 37° C. and for a period in the range of from 12 hours to 36 hours, e.g. 24 hours. Weekly subculturing of bacteria into MYGP may be performed to maintain activity, if desired. MYGP media may be obtained, for example, from Oxoid, Ltd.

In some embodiments, multiple iterations of subculturing the Kluyveromyces marxianus are carried out prior to using the strain to re-ferment the at least partially hydrolysed peptide composition. In some embodiments, prior to use in the re-fermentation step, the Kluyveromyces marxianus is activated by three iterations of incubating in MYGP medium at about 37° C. for 12 to 18 hours, and replacing at least a portion of the MYGP medium. In some embodiments, the media in which the yeast is activated is exchanged for a milk composition (e.g. pasteurised RSM) prior to adding the Kluyveromyces marxianus to the at least partially hydrolysed peptide composition and carrying out a second fermentation step.

The further quantity of milk composition may for example be a milk composition as described above, for example it may be reconstituted skimmed milk. In some embodiments, the further quantity of milk composition is reconstituted skimmed milk having from 8% to 16% w/v skim milk powder, for example about 12% w/v skim milk powder, admixed with distilled water.

In some embodiments the further milk composition is the same type of milk composition as used in step a).

The further quantity of milk composition may for example be pasteurised. In some embodiments the further quantity of milk composition is pasteurised reconstituted skimmed milk.

In some embodiments, the further quantity of milk composition is 12% w/v reconstituted skimmed milk which has been pasteurised by heating it at a temperature in the range of from 85° C. to 95° C. for a period in the range of from 15 minutes to 18 minutes.

The further quantity of milk composition may for example be subjected to mechanical agitation prior to admixing with the hydrolysed peptidic composition and Kluyveromyces marxianus. The further quantity of milk composition may be mechanically agitated under conditions as described above. For example, shaking of the vessel containing the further quantity of milk composition may be used. Alternatively, a stirrer or impeller may be used. An agitation rate of from 100 rpm to 600 rpm may be used, for example. In some embodiments, the further quantity of milk composition is agitated at a rate in the range of from 100 rpm to 600 rpm, or from 150 rpm to 400 rpm, or from 200 rpm to 300 rpm, or from 200 rpm to 225 rpm. The mechanical agitation step may for example be carried out for a time period in the range of from 15 minutes to 3 hours, from 30 minutes to 2 hours, or from 30 minutes to 1 hour. The mechanical agitation step can be conducted at any suitable temperature. In some embodiments, the mechanical agitation step is carried out at about 37° C.

In some embodiments, the further quantity of milk composition is 12% w/v reconstituted skimmed milk which has been pasteurised by heating it at a temperature in the range of from 85° C. to 95° C. for a period in the range of from 15 minutes to 18 minutes, and which has been mechanically agitating at a temperature of about 37° C. for a period in the range of from 30 minutes to 1 hour.

In some embodiments, the further quantity of milk composition is admixed with hydrolysed peptide composition following step b) without significant additional processing of the hydrolysed peptide composition, for example the further quantity of milk composition may be added following cooling of the product of step b), e.g. to 37° C.

The amount of further milk composition which is admixed with the hydrolysed peptide composition may for example be in the range of from 5% v/v to 20% v/v of the hydrolysed peptide composition, e.g. about 10% v/v of the hydrolysed peptide composition.

A suitable amount of Kluyveromyces marxianus is added. For example, in some embodiments, an amount of a Kluyveromyces marxianus composition is added which is in the range of from 0.5 to 5% of the volume of the product of step b), or which is in the range of from 0.5% v/v to 3% v/v, or which is in the range of from 1% to 2% v/v, or which is about 1% v/v.

Typically, the cell count of the composition containing the activated yeast, which is added to the hydrolysed peptide composition, is in the range of from 10⁶ to 10¹⁰ CFU/mL. In some embodiments, prior to addition to the hydrolysed peptide composition, the concentration of yeast in the activated culture is in the range of from 10⁷ to 10⁹ CFU/mL. In some embodiments, the concentration of yeast in the activated culture is about 10^{7.9} CFU/mL.

Other fermentation conditions used may for example be similar to those used in step a). For example, in some embodiments the fermentation step with Kluyveromyces marxianus is carried out for a period in the range of from 4 to 36 hours, or 4 to 24 hours, or from 8 to 12 hours, or about 8 hours. Typically, the fermentation step with Kluyveromyces marxianus is carried out at a temperature of about 37° C., although any suitable temperature may be used. Preferably, the temperature is maintained at a constant level during the fermentation step.

In some embodiments, the admixture of hydrolysed peptide composition, further milk composition and Kluyveromyces marxianus is fermented for a period in the range of from 8 to 12 hours, with agitation, in a bioreactor, bio-fermentation apparatus or using an incubator.

In some embodiments, following fermentation with Kluyveromyces marxianus, the product is heat treated, in order to inactivate the Kluyveromyces marxianus. The heat treatment conditions also substantially avoiding denaturing or otherwise adversely affecting the antihypertensive hydrolysed milk peptides. Typically, the heat treatment step involves subjecting the product of the Kluyveromyces marxianus fermentation step to a temperature in the range of from 80 to 90° C. for a suitable period of time, e.g. in the range of from 10 minutes to 25 minutes. In some embodiments, the product is subjected to a temperature in the range of from 80° C. to 90° C. for a period in the range of from 6 to 30 minutes, or from 12 to 15 minutes. In some embodiments, the product is subjected to a temperature of about 85° C. for a period in the range of from 12 to 15 minutes.

Once the heat-treatment step is complete, the peptide composition may be cooled, e.g. to room temperature, and may be subjected to further processing.

In some embodiments, following heat treatment of the product of the Kluyveromyces marxianus fermentation step, antihypertensive peptide is separated from other components of the mixture. For example, in some embodiments, antihypertensive peptide is separated from Kluyveromyces marxianus. In some embodiments, antihypertensive peptide is separated using centrifugation.

The antihypertensive peptidic composition resulting from the double fermentation process may be obtained and used in any suitable form, for example as a liquid composition, or as a dry powder. In some embodiments, following the step of fermenting with Kluyveromyces marxianus, the antihypertensive peptide composition is lyophilised or freeze-dried, i.e. to produce a solid form of the composition.

It has been found that the antihypertensive peptide composition obtained following the double fermentation process described above has high ACE inhibitory activity, as set out in the Examples below. For example, following the protocol of Donkor and others (2007a), lyophilised 40 mg samples of peptide composition were found to have high activity in inhibiting hippuric acid release from hippuryl-histidyl-leucine by ACE. In some embodiments, when tested according to the protocol set out in the examples, the composition achieves at least 98% inhibition of hippuric acid release, or 100% inhibition of hippuric acid release.

In the second aspect, the process involves:
i) subjecting a milk composition to mechanical agitation;
ii) fermenting the product of step i) with a Lactobacillus helveticus and a peptidase selected from the group consisting of an aminopeptidase and a serine endopeptidase, thereby at least partially hydrolysing peptides present in the composition; and
iii) heat-treating the product of step ii) so as to inactivate the Lactobacillus helveticus and the peptidase.

Any suitable milk composition may be used. In some embodiments, the milk composition is milk, for example semi-skimmed or skimmed milk. For example, it may be a milk composition having 3-5 g/100 g protein, 0.05-0.2 g/100 g fat, and 4-7 g/100 g sugars, or it may be a milk composition having 3.5 g/100 g protein, 0.1 g/100 g fat, and 5.3 g/100 g sugars. In some embodiments, the milk composition is a whey protein isolate or concentrate. In some embodiments, the milk composition is a reconstituted milk composition, such as that produced by admixing appropriate quantities of milk powder with water, e.g. distilled water. In some embodiments, the milk composition is a reconstituted semi-skimmed milk. In some embodiments, the milk composition is reconstituted skimmed milk (RSM). For example, it may be reconstituted skimmed milk produced from a powder composition containing 40-70% sugars (e.g. lactose), 30-50% protein, and 0.5-2% fat, or from a powder composition containing 52% sugars (e.g. lactose), 37% protein and 1.2% fat. In some embodiments, the milk composition is reconstituted skimmed milk having from 8% to 16% w/v skim milk powder, for example about 12% w/v skim milk powder, admixed with distilled water. In some embodiments, the milk composition is reconstituted whey protein concentrate, e.g. having from 2 to 6% w/v whey protein concentrate, for example 4% w/v whey protein concentrate, admixed with distilled water.

The milk composition has typically been treated prior to use to at least partially sterilise the composition. In some embodiments, the milk composition is a pasteurised milk composition, for example a pasteurised reconstituted skimmed milk composition. Pasteurisation is a technique used for partial sterilization of a product such as milk, involving heating for a suitable period of time, typically at a temperature of less than 100° C.

Pasteurisation may be carried out as part of the process, for example as a step of the process. Alternatively, pasteurisation of the milk composition may be carried out separately from carrying out the main process. For example pasteurisation of a milk composition may be carried out at a farm or a dairy plant, prior to shipping of the pasteurised milk composition to the site where the process is carried out.

In some embodiments, the milk composition is pasteurised by heating it at a temperature in the range of from 85° C. to 95° C., for example at a temperature of about 90° C. In some embodiments, prior to mechanical hydrolysis, the milk composition is pasteurised by heating it for a period in the range of from 15 seconds to 1 hour, for example for a period in the range of from 1 minute to 30 minutes, from 5 minutes to 30 minutes, from 10 minutes to 30 minutes, from 20 minutes to 30 minutes, from 5 minutes to 20 minutes, from 10 minutes to 20 minutes, from 15 minutes to 20 minutes, from 15 minutes to 18 minutes, or about 15 minutes.

In some embodiments, the milk composition is pasteurised by heating it at a temperature in the range of from 85° C. to 95° C. for a period in the range of from 15 minutes to 18 minutes, for example by heating it at about 90° C. for a period of about 15 minutes.

Step i) of the process of the second aspect involves subjecting the milk composition to mechanical agitation. As discussed above, it is believed that the use of mechanical agitation results in partial hydrolysis of peptides, and facilitates the production of improved hydrolysed peptidic compositions having particularly high levels of antihypertensive activity. Any suitable means of agitating the milk composition may be employed. For example, shaking of the vessel containing the pasteurised milk composition may be used. Alternatively, a stirrer or impeller may be used. An agitation rate of from 100 rpm to 600 rpm may be used, for example. In some embodiments, the milk composition is agitated at a rate in the range of from 100 rpm to 600 rpm, or from 150 rpm to 400 rpm, or from 200 rpm to 300 rpm, or from 200 rpm to 225 rpm. The mechanical agitation step may for example be carried out for a time period in the range of from 15 minutes to 3 hours, from 30 minutes to 2 hours, or from 30 minutes to 1 hour. The mechanical agitation step can be conducted at any suitable temperature. In some embodiments, the mechanical hydrolysis step is carried out at about 37° C.

In some embodiments, mechanical agitation is carried out by agitating the milk composition at a temperature of about 37° C. for a period in the range of from 30 minutes to 1 hour. In some embodiments, mechanical agitation is carried by agitating the milk composition at a rate in the range of from 200 to 225 revolutions per minute, at a temperature of about 37° C., and for a period in the range of from 30 minutes to 1 hour.

Step ii) involves fermenting the milk composition with a Lactobacillus helveticus and a peptidase, thereby at least partially hydrolysing peptides present in the composition, e.g. to produce a composition containing high levels of tripeptides such as Val-Pro-Pro and Ile-Pro-Pro.

Any suitable strain of Lactobacillus helveticus may be utilised in the present process. However, in some preferred embodiments, the Lactobacillus helveticus is Lactobacillus helveticus 881315, Lactobacillus helveticus 881188, or a mixture of the two. As discussed above, fermentation of the milk composition using those strains of bacteria has been found to result in production of fermented peptide composition having high antihypertensive properties. Lactobacillus helveticus 881315 and 881188 may be obtained from commercial sources, for example from Dairy Innovation Australia Ltd (now Chr. Hansen Ltd.) Werribee, VIC, Australia.

Prior to the fermentation step, the Lactobacillus helveticus strain or strains is/are activated, for example by transferring an aliquot of the strain into a suitable broth (e.g. Man, Rogosa, and Sharpe (MRS) broth) and incubating, e.g. at 37° C. and for a period in the range of from 12 hours to 24 hours. Any suitable broth or media may be used. In some embodiments, a dairy media is used. In some embodiments, MRS broth is used. MRS broth may be obtained for example from Sigma Aldrich. In some embodiments, multiple iterations of subculturing the Lactobacillus helveticus are carried out in order to activate the bacteria, prior to using the strain to ferment the milk composition. In some embodiments, prior to step ii), the Lactobacillus helveticus is activated by three iterations of incubating in MRS medium at about 37° C. for 12-18 hours, and replacing at least a portion of the MRS medium. In some embodiments, the media in which the bacteria is activated is exchanged for a milk composition (e.g. pasteurised reconstituted skimmed milk) prior to adding the Lactobacillus helveticus to the milk composition to be fermented and carrying out step ii).

Typically, the cell count of the composition containing the activated bacteria, which is added to the milk composition, is in the range of from 10⁷ to 10¹¹ CFU/mL. Following addition to the milk composition, the Lactobacillus helveticus will typically be present at a cell count in the range of from 10⁵ to 10⁹ CFU/mL. In some embodiments, prior to addition to the milk composition, the concentration of bacteria in the activated culture is in the range of from 10⁸ to 10¹¹ CFU/mL, or in the range of from 10⁸ to 10⁹ CFU/mL. In some embodiments, the concentration of bacteria in the activated culture is about 10¹¹ CFU/mL, or about 10^{10.8} CFU/mL.

A peptidase selected from the group consisting of an aminopeptidase and a serine endopeptidase is also used.

In some embodiments, the peptidase is an aminopeptidase. In some embodiments, the aminopeptidase is a leucine aminopeptidase (i.e. an aminopeptidase which preferentially cleaves leucine residues. In some embodiments, the aminopeptidase preferentially cleaves the N-terminal amino acid from a sequence Xaa-Yaa, in which Xaa is preferably Leu or Pro, and Yaa is Pro.

In some embodiments the aminopeptidase enzyme is a fungal protease/peptidase complex produced by submerged fermentation of a strain of Aspergillus oryzae which contains both endoprotease and exopeptidase activities. In some embodiments, the aminopeptidase is present in the form of a composition having an activity in the range of from 800-1200 leucine amino-peptidase (LAPU g⁻¹), or about 1000 leucine amino-peptidase (LAPU g⁻¹), for example such as the Flavourzyme® 1000L product (EC 3.4.11.1) available from Novozymes Australia, NSW, Australia.

In some embodiments, the peptidase is a serine endopeptidase, for example a subtilisin. For example a serine endopeptidase obtained from Bacillus licheniformis may be used, and/or for example present in the form of a composition having an activity of at least 1 Anson units per gram, or at least 2 Anson units per gram, or at least 2.4 Anson units per gram, or in the range of from 1 to 5 Anson units per gram. One such example is the product sold under the trade name Alcalase®, available from Sigma-Aldrich.

A suitable amount of Lactobacillus helveticus and a suitable amount of peptidase are added to the milk composition.

For example, in some embodiments, an amount in the range of from 0.5 to 5% v/v of a composition comprising the Lactobacillus helveticus is added, or an amount in the range of from 0.5% to 3% v/v, or an amount in the range of from 1% to 2% v/v, or an amount which is about 1% v/v.

In some embodiments, an amount in the range of from 0.02 to 1% w/w of a peptidase composition is added to the milk composition, or an amount in the range of from 0.04 to 0.45% w/w is added, or an amount of about 0.14 w/w%.

In some embodiments, an amount of an aminopeptidase composition having an activity in the range of from 800-1200 LAPU g⁻¹, and which is in the range of from 0.02 to 1% w/w of the milk composition is added.

In some embodiments, an amount of an aminopeptidase composition having an activity in the range of from 800-1200 LAPU g⁻¹, and which is in the range of from 0.04 to 0.45% w/w of the milk composition is added, e.g. about 0.14 w/w%.

In some embodiments, to commence fermentation, an amount in the range of from 1 to 2% v/v of a composition comprising the Lactobacillus helveticus, and an amount in the range of from 0.04 to 0.45% w/w of a peptidase composition is added to the milk composition.

In some embodiments, to commence fermentation, an amount in the range of from 1 to 2% v/v of a composition comprising the Lactobacillus helveticus present at a concentration in the range of from 10⁸ to 10⁹ CFU/mL, and an amount in the range of from 0.04 to 0.45% w/w of an aminopeptidase composition having an activity in the range of from 800-1200 LAPU g⁻¹, is added to the milk composition.

The fermentation step is carried out for a period suitable to effect high levels of hydrolysis of the peptides in the milk composition. For example, in some embodiments the fermentation step is carried out for a period in the range of from 4 to 36 hours, or 4 to 24 hours, or from 8 to 12 hours, or about 12 hours. Typically, the fermentation step is carried out at a temperature of about 37° C., although any suitable temperature may be used. Preferably, the temperature is maintained at a constant level during the fermentation step.

Typically, the fermentation step generates quantities of lactic acid, such that the pH of the mixture decreases over the course of the fermentation step. Preferably, the initial pH of the fermentation step is in the range of from 6 to 7, for example 6.3 to 6.7, or about 6.5. By the end of the fermentation, the final pH may have decreased for example to a pH in the range of from 3 to 4, for example in the range of from 3.3 to 3.5, or at about pH 3.4.

The fermentation step may be carried out in a suitable bioreactor, bio-fermentation apparatus, or using an incubator. The bioreactor may for example contain a thermal jacket, an agitation system (e.g. including an impeller or other means for stirring/agitating the mixture), and may contain sensors/probes for monitoring conditions such as temperature and pH. An example of a suitable bioreactor is the Biostat® A plus bioreactor. Typically, the mixture is agitated/stirred during the fermentation step. In some embodiments, the fermentation step is carried out with agitation at a rate in the range of from 100 to 500 revolutions per minute, or from 150 to 300 revolutions per minute, or from 200 to 225 revolutions per minute.

Step iii) of the process involves heat-treating the product of step ii). The heat treatment is sufficient to inactivate the Lactobacillus helveticus and the peptidase. The heat treatment also substantially avoiding denaturing or otherwise adversely affecting the quality of the hydrolysed milk peptides. Typically, the heat treatment step involves subjecting the product of step ii) to a temperature in the range of from 80 to 90° C. for a suitable period of time, e.g. in the range of from 10 minutes to 25 minutes. In some embodiments, in step iii) the composition is subjected to a temperature in the range of from 80° C. to 90° C. for a period in the range of from 12 to 15 minutes. In some embodiments, in step iii) the composition is subjected to a temperature of about 85° C. for a period in the range of from 12 to 15 minutes.

Once the heat-treatment step is complete, the peptidic composition may for example be cooled to room temperature, and may be subjected to further processing.

In some embodiments, following step iii), peptide is separated from other components of the mixture. For example, in some embodiments, following step iii), peptide is separated from Lactobacillus helveticus. In some embodiments, following step iii), peptide is separated from peptidase. Any suitable separation technique may be used, for example such as centrifugation. However, for some applications, it may not be necessary to separate the hydrolysed peptide from other components of the mixture. Thus, in some embodiments, following step iii) the peptide composition may be used without separation or purification.

The antihypertensive peptide composition may be obtained and used in any suitable form, for example as a liquid composition, or as a dry powder. In some embodiments, following step iii), the antihypertensive peptide composition is lyophilised, i.e. to produce a solid form of the composition.

It has been found that the peptide composition obtained by the process of the second aspect has high ACE inhibitory activity, as set out in the Examples below. For example, following the protocol of Donkor and others (2007a), lyophilised 40 mg samples of hydrolysed peptidic composition were found to have high activity in inhibiting hippuric acid release from hippuryl-histidyl-leucine by ACE. In some embodiments, when tested according to the protocol set out in the examples, the composition achieves at least 95% inhibition of hippuric acid release.

In some embodiments, following step iii), the at least partially hydrolysed peptides are admixed with a further quantity of milk composition and with Kluyveromyces marxianus, and the mixture is fermented to produce the antihypertensive peptide composition. As discussed above, it has also been found that, by carrying out further fermentation of the at least partially hydrolysed peptide composition, compositions having further improved ACE inhibitory activity can be obtained.

Any suitable Kluyveromyces marxianus may be used. In some embodiments, the Kluyveromyces marxianus is Kluyveromyces marxianus LAF4. Kluveromyces marxianus LAF4 may for example be obtained from Dairy Innovation Australia Ltd (now Chr. Hansen Ltd.) Werribee, VIC, Australia.

Prior to the second fermentation step, the Kluyveromyces marxianus may be activated, for example by transferring an aliquot of the strain into a suitable medium, e.g. sterile MYGP medium (a medium containing malt extract, yeast extract, glucose, peptone) and incubating, e.g. at 37° C. and for a period in the range of from 12 hours to 36 hours, e.g. 24 hours. Weekly subculturing of bacteria into MYGP may be performed to maintain activity, if desired. MYGP media may be obtained, for example, from Oxoid, Ltd.

In some embodiments, multiple iterations of subculturing the Kluyveromyces marxianus are carried out prior to using the strain to re-ferment the at least partially hydrolysed peptide composition. In some embodiments, prior to use in the re-fermentation step, the Kluyveromyces marxianus is activated by three iterations of incubating in MYGP medium at about 37° C. for 12 to 18 hours, and replacing at least a portion of the MYGP medium. In some embodiments, the media in which the yeast is activated is exchanged for a milk composition (e.g. pasteurised RSM) prior to adding the Kluyveromyces marxianus to the at least partially hydrolysed peptide composition and carrying out a second fermentation step.

The further quantity of milk composition may for example be a milk composition as described above, for example it may be reconstituted skimmed milk. In some embodiments, the further quantity of milk composition is reconstituted skimmed milk having from 8% to 16% w/v skim milk powder, for example about 12% w/v skim milk powder, admixed with distilled water.

In some embodiments the further milk composition is the same type of milk composition as used in step i).

The further quantity of milk composition may for example be pasteurised. In some embodiments the further quantity of milk composition is pasteurised reconstituted skimmed milk.

In some embodiments, the further quantity of milk composition is 12% w/v reconstituted skimmed milk which has been pasteurised by heating it at a temperature in the range of from 85° C. to 95° C. for a period in the range of from 15 minutes to 18 minutes.

The further quantity of milk composition may for example be subjected to mechanical agitation prior to admixing with the hydrolysed peptidic composition and Kluyveromyces marxianus. The further quantity of milk composition may be mechanically agitated under conditions as described above. For example, shaking of the vessel containing the further quantity of milk composition may be used. Alternatively, a stirrer or impeller may be used. An agitation rate of from 100 rpm to 600 rpm may be used, for example. In some embodiments, the further quantity of milk composition is agitated at a rate in the range of from 100 rpm to 600 rpm, or from 150 rpm to 400 rpm, or from 200 rpm to 300 rpm, or from 200 rpm to 225 rpm. The mechanical agitation step may for example be carried out for a time period in the range of from 15 minutes to 3 hours, from 30 minutes to 2 hours, or from 30 minutes to 1 hour. The mechanical agitation step can be conducted at any suitable temperature. In some embodiments, the mechanical agitation step is carried out at about 37° C.

In some embodiments, the further quantity of milk composition is 12% w/v reconstituted skimmed milk which has been pasteurised by heating it at a temperature in the range of from 85° C. to 95° C. for a period in the range of from 15 minutes to 18 minutes, and which has been mechanically agitating at a temperature of about 37° C. for a period in the range of from 30 minutes to 1 hour.

In some embodiments, the further quantity of milk composition is admixed with hydrolysed peptide composition following step iii) without significant additional processing of the hydrolysed peptide composition, for example the further quantity of milk composition may be added following cooling of the product of step iii), e.g. to 37° C.

The amount of further milk composition which is admixed with the hydrolysed peptide composition may for example be in the range of from 5% v/v to 20% v/v of the hydrolysed peptide composition, e.g. about 10% v/v of the hydrolysed peptide composition.

A suitable amount of Kluyveromyces marxianus is added. For example, in some embodiments, an amount of a Kluyveromyces marxianus composition is added which is in the range of from 0.5 to 5% of the volume of the product of step iii), or which is in the range of from 0.5% v/v to 3% v/v, or which is in the range of from 1% to 2% v/v, or which is about 1% v/v.

Typically, the cell count of the composition containing the activated yeast, which is added to the hydrolysed peptide composition, is in the range of from 10⁶ to 10¹⁰ CFU/mL. In some embodiments, prior to addition to the hydrolysed peptide composition, the concentration of yeast in the activated culture is in the range of from 10⁷ to 10⁹ CFU/mL. In some embodiments, the concentration of yeast in the activated culture is about 10^{7.9} CFU/mL.

Other fermentation conditions used may for example be similar to those used in step ii). For example, in some embodiments the fermentation step with Kluyveromyces marxianus is carried out for a period in the range of from 4 to 36 hours, or 4 to 24 hours, or from 8 to 12 hours, or about 8 hours. Typically, the fermentation step with Kluyveromyces marxianus is carried out at a temperature of about 37° C., although any suitable temperature may be used. Preferably, the temperature is maintained at a constant level during the fermentation step.

In some embodiments, the admixture of hydrolysed peptide composition, further milk composition and Kluyveromyces marxianus is fermented for a period in the range of from 8 to 12 hours, with agitation, in a bioreactor, bio-fermentation apparatus or using an incubator.

In some embodiments, following fermentation with Kluyveromyces marxianus, the product is heat treated, in order to inactivate the Kluyveromyces marxianus. The heat treatment conditions also substantially avoiding denaturing or otherwise adversely affecting the antihypertensive hydrolysed milk peptides. Typically, the heat treatment step involves subjecting the product of the Kluyveromyces marxianus fermentation step to a temperature in the range of from 80 to 90° C. for a suitable period of time, e.g. in the range of from 10 minutes to 25 minutes. In some embodiments, the product is subjected to a temperature in the range of from 80° C. to 90° C. for a period in the range of from 6 to 30 minutes, or from 12 to 15 minutes. In some embodiments, the product is subjected to a temperature of about 85° C. for a period in the range of from 12 to 15 minutes.

Once the heat-treatment step is complete, the peptide composition may be cooled, e.g. to room temperature, and may be subjected to further processing.

In some embodiments, following heat treatment of the product of the Kluyveromyces marxianus fermentation step, antihypertensive peptide is separated from other components of the mixture. For example, in some embodiments, antihypertensive peptide is separated from Kluyveromyces marxianus. In some embodiments, antihypertensive peptide is separated using centrifugation.

The antihypertensive peptidic composition resulting from the double fermentation process may be obtained and used in any suitable form, for example as a liquid composition, or as a dry powder. In some embodiments, following the step of fermenting with Kluyveromyces marxianus, the antihypertensive peptide composition is lyophilised or freeze-dried, i.e. to produce a solid form of the composition.

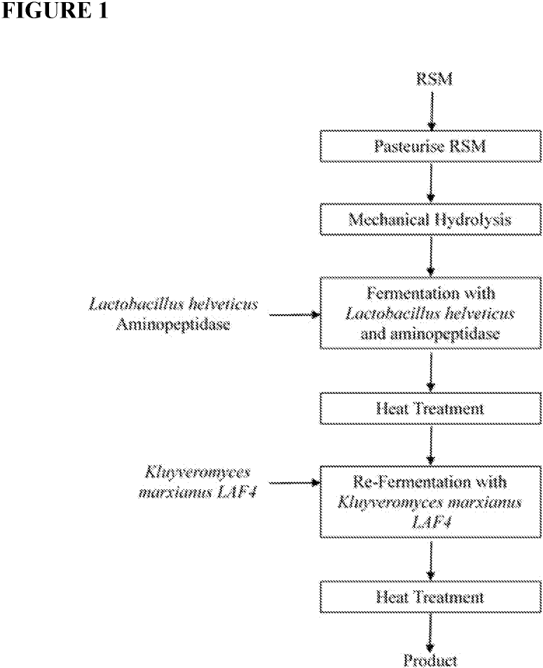

An embodiment of a process in accordance with the present disclosure is shown in FIG. 1. In that embodiment, 12% reconstituted skimmed milk is pasteurised, by heating it at a temperature in the range of from 85° C. to 95° C. for a period of about 15 minutes. The pasteurised skimmed milk is the subject to mechanical agitation, by shaking the milk composition at a rate of about 200 rpm, at 37° C., for a period of about 45 minutes.

Separately, Lactobacillus helveticus is activated by carrying out three iterations of incubating in MRS medium at about 37° C. for about 18 hours. Following activation the Lactobacillus helveticus composition has an activity of about 10^{10.8} CFU/mL. An amount of 1% v/v of the Lactobacillus helveticus composition is added to the milk composition, together with 0.14% w/w Flavourzyme® 1000 L. The resulting mixture is fermented for a period of 10 hours at 37° C.

Following fermentation, the mixture is heated to a temperature of 85° C. for 15 minutes to deactivate the Lactobacillus helveticus and the peptidase.

A further quantity of mechanically agitated pasteurised 12% reconstituted skimmed milk is then added, at 10% of the volume of the fermented mixture. A Kluyveromyces marxianus composition, which has been activated by three iterations of incubating in MYGP medium at about 37° C. for about 18 hours, such that the concentration of yeast in the activated culture is about 10^{7.9} CFU/mL, is added to the fermented mixture in an amount of 1% of the volume of the fermented mixture.

The resulting mixture is fermented for a period of 10 hours at 37° C.

Following fermentation, the mixture is heated to a temperature of 85° C. for 15 minutes to deactivate the Kluyveromyces marxianus.

Following the heat treatment, the antihypertensive peptide composition is separated from the inactivated yeast and bacteria, by centrifugation. The peptide composition is then freeze-dried to provide the product as a powder.

As discussed above, the processes produce novel peptide compositions having unexpectedly high ACE inhibitory properties. Thus, in another aspect, there is also provided an antihypertensive peptide composition producible according to a process as defined herein.

The fermented peptide compositions have ACE-inhibitory activity. The peptide compositions are also understood to have antioxidant activity (Rajapakse et al., 2005; Unal & Akalin, 2012), inhibit growth of pathogenic bacteria, and reduce the risk of cancer (Kawase et al, 2000; Alhaj et al., 2007; Fitzgerald et al., 2011; Marinik et al., 2013; Guo et al., 2015). Studies have also suggested the involvement of the renin angiotensin system in obesity related to hypertension (Hall et al., 2000; Boustany et al., 2004). Studies have also shown that oral administration of peptides produced by fermentation of dairy proteins may affect the cardiovascular, digestive, immune and nervous systems (Erdmann, Cheung, & Schroder, 2008; FitzGerald et al., 2011; Yamamoto et al., 2010). Other studies on fermented milk products have reported on those products being responsible for health benefits for consumers (Shah, 2007; López-Expósito, Amigo, & Recio, 2012; Hernandez-Ledesma et al., 2014). Health benefits of regular consumption of milk products containing probiotics have been widely reported and include development of intestinal microbial balance, reduction of risk of colon cancer, protection against breast cancer, strengthening the immune system, lowering blood pressure and blood cholesterol levels, reduction in some forms of food allergies, and inhibiting the growth of pathogenic bacteria (Kawase et al., 2000; Alhaj et al., 2007; Fitzgerald et al., 2011; Marinik et al., 2013; Guo et al., 2015). Most recently, it has been shown that bioactive peptides can be used to protect patients against infections with SARS-CoV-2 (Matsoukas et al. 2021), and may be able to prevent the development of the pathological process during COVID-19 by inhibiting SARS-CoV-2 virus proteins (Khavinson et al. 2020). Accordingly, in some embodiments, the peptide composition has antioxidant activity, immune-enhancing activity, antibacterial activity, antiviral activity, anticancer activity, antihypertensive activity and/or facilitates weight loss.

The peptide composition may be provided in any suitable form, e.g. in a pharmaceutical dosage form, as a food or beverage to which the peptide composition has been added, or as a powder supplement for adding to food/drink. Thus, in another aspect, there is also provided a dosage form comprising the antihypertensive peptide composition as defined herein.

The dosage form may for example be in the form of a liquid, powder, tablet or capsule, which contains the peptide composition, together with one or more pharmaceutically acceptable excipients. Suitable excipients are known to the person skilled in the art and, depending on the dosage form, may for example include carriers, fillers, binders, disintegrants, antioxidants, preservatives, lubricants, glidants, anti-caking agents, suspending agents, solvents, capsule shell excipients.

In another aspect, there is also provided a food product or supplement comprising the antihypertensive peptide composition as defined herein. For example, the peptide composition (e.g. in solid or liquid form) may be incorporated into a food product. In some embodiments, the food product is a spread or a yogurt. The peptide composition may also be provided in the form of a dietary supplement, e.g. a powder for adding to food or drink, or in the form of a capsule or tablet for taking alongside a normal diet.

In another aspect, there is also provided a powdered milk formula comprising the antihypertensive peptide composition as defined herein. Such a composition will typically contain a conventional powdered milk, together with a suitable amount of the antihypertensive hydrolysed peptide composition in solid (e.g. powder) form.

In another aspect, there is also provided a beverage comprising the antihypertensive peptide composition as defined herein. In some embodiments, the beverage is a milk drink.

In some embodiments, the dosage form, food product, food supplement, powdered milk formula or beverage as defined herein comprises one or more of a flavouring, antioxidant and sweetener. For example, in order to improve the long term storage stability of compositions containing the fermented peptides, an antioxidant may be included to prevent or reduce oxidation. Sweeteners and flavourings may be included in products containing the peptide composition, for example in order to improve palatability. Natural sweeteners, such as sucrose, or stevia sweeteners may be used. Alternatively, or in addition, artificial sweeteners such as saccharin, aspartame, acesulfame K may be utilised. Examples of suitable flavourings include blueberry flavouring, for example such as the FlavDrops™ product, a natural zero-calorie flavouring.

It will be appreciated that the antihypertensive peptide composition, which has antihypertensive properties, may be used therapeutically and prophylactically, e.g. in connection with those diseases, disorders and conditions for which administration of antihypertensives may be beneficial. This includes, for example, treating, preventing, or lessening the severity of one or more symptoms associated with such a disease, disorder or condition. Examples of such conditions include cardiovascular diseases, disorders and conditions, hypertension, heart disease, being overweight or obese, bacterial infections and cancer.

Accordingly, in another aspect, there is provided a method of treating, preventing, or reducing the likelihood of a disease or disorder in a subject, comprising administering an effective amount of an antihypertensive peptide composition, dosage form, food product, food supplement, powdered milk formula or beverage as defined herein to the subject.

There is also provided an antihypertensive peptide composition, dosage form, food product, powdered milk formula or beverage as defined herein, for use in the treatment or prevention of, or for use in reducing the likelihood of, a disease or disorder.

In some embodiments, the disease or disorder is a cardiovascular disease or disorder, a bacterial infection, or cancer. In some embodiments, the disease or disorder is selected from the group consisting of hypertension and heart disease. In some embodiments, the disease or disorder is being overweight or obese.

There is also provided use of an antihypertensive peptide composition, dosage form, food product, food supplement, powdered milk formula or beverage as defined herein, for the manufacture of a medicament, for the treatment, prevention, or for reducing the likelihood of a disease or disorder selected from the group consisting of a bacterial infection, cancer, a cardiovascular disease, hypertension, heart disease, being overweight, and obesity.

In some embodiments, the subject is an animal, e.g. a mammal. In some embodiments the subject is a human. In some embodiments the subject is an adult human. In other embodiments the subject is a child. In some embodiments, the subject is a non-human animal, e.g. a non-human mammal.

The dose or frequency of administration may for example be dependent on factors such as the age, weight, general physical condition of the subject, or other clinical symptoms specific to the subject to be treated, and can be determined by one of skill in the art. For example, in some embodiments, the antihypertensive peptide composition, or dosage form, food product, food supplement, powdered milk formula or beverage containing the peptide composition, may be administered as needed, e.g. once, twice, or three times per week, once every other day, once per day, or twice per day, or three times per day, or four times per day for example. Any suitable dosage of the antihypertensive peptide composition, or dosage form, food product, food supplement, powdered milk formula or beverage containing the peptide composition, may be administered, for example a dosage in the range of from 1 mg to 50 g, from 1 mg to 20 g, from 1 mg to 10 g, from 1 mg to 5 g, from 1 mg to 3 g, from 1 mg to 2 g, from 1 mg to 1 g, from 1 mg to 500 mg, from 1 mg to 250 mg, from 1 g to 50 g, from 2 g to 50 g, from 5 g to 50 g, from 10 g to 50 g, from 20 g to 50 g, from 1 g to 20 g, or from 2 g to 10 g.

It will be appreciated by persons skilled in the art that numerous variations and/or modifications may be made to the above-described embodiments, without departing from the broad general scope of the present disclosure. The present embodiments are, therefore, to be considered in all respects as illustrative and not restrictive.

### EXAMPLES

The following examples are to be understood as illustrative only. They should therefore not be construed as limiting the invention in any way.

### Example 1: Preparation of Fermentation Substrate

For producing antihypertensive peptide compositions, 12% w/v reconstituted skimmed milk (RSM) was pasteurised and subjected to mechanical hydrolysis, as detailed below, prior to the subsequent fermenting step.

Skim milk powder (Murray Goulburn Co-operative Co. Ltd, Brunswick, VIC Australia) (52% lactose, 37% protein, 8.6% ash and 1.2% fat) was reconstituted using distilled water. Reconstituted medium (12% w/v RSM, 5 L) was pasteurized by heating at 90° C. in a water bath for 15-18 min. The resulting medium was cooled to 37° C. then shaken (200 rpm) for 30-60 min at 37° C. to effect mechanical hydrolysis.

### Example 2: Fermentation Organisms

For producing antihypertensive peptide compositions by fermentation, strains of Lactobacillus helveticus were propagated as detailed below. For experiments involving further fermentation with yeast, Kluyveromyces marxianus was also propagated as described below.

Lactobacillus helveticus strains ASCC 881315 and 881188 were obtained from the Australian Starter Culture Collection (Dairy Innovation Australia Ltd., Werribee, Victoria, Australia), and stored in sterile de Man, Rogosa, and Sharpe (MRS) broth containing 40% glycerol (Oxoid, Ltd., West Heidelberg, Victoria, Australia) at -80° C. For activation, an aliquot (100 µL) of each strain was individually transferred into sterile MRS broth (10 mL) and incubated at 37° C. for 24 h. Weekly subculturing of bacteria into the MRS broth was performed to maintain the bacterial activity. Prior to production of hydrolysed peptidic compositions by fermentation, each strain was cultured three times in MRS broth. Thus, 10 mL aliquots of MRS were inoculated with 1% (v/v) of the above bacterial culture and incubated at 37° C. for 12 h. After a total of three successive incubations in MRS, the bacterial cultures (10⁸-10⁹ CFU/mL) were ready to be used in the fermentation process. Alternatively, the cultures were used to inoculate 10 mL aliquots of 12% RSM from Example 1 and incubated at 37° C. for 12 h prior to being used in the subsequent fermentation process.

Kluyveromyces marxianus LAF4 was obtained from Chr. Hansen Pty Ltd. For activation, an aliquot (100 µL) of the strain was transferred into 10 mL of sterile MYGP medium (a medium containing malt extract, yeast extract, glucose, peptone) (Sigma-Aldrich Australia), and incubated at 37° C. for 24 h. MYGP medium contained, per liter purified filtered water: 3 g yeast extract, 10 g dextrose; 3 g malt extract, 5 g gelatin peptone, 20 g agar, 0.4 g cupric sulfate, and had pH 6.2. Weekly subculturing of yeast into MYGP was performed to maintain the activity. Prior to production of antihypertensive peptide compositions by fermentation, K. marxianus LAF4 was cultured three times in MYGP. Thus, 10 mL aliquots of MYGP were inoculated with 1% (v/v) of the above yeast culture and incubated at 37° C. for 12 h. After a total of three successive transfers in MYGP, the yeast cultures were ready to be used in the fermentation process. Alternatively, the cultures were used to inoculate 10 mL aliquots of 12% RSM from Example 1 and incubated at 37° C. for 12 h prior to being used in the subsequent fermentation process.

### Example 3: Fermentation of Pre-Treated Milk Composition Substrate With L helveticus 881315 and Flavourzyme®

For producing antihypertensive peptide compositions, pasteurized and mechanically hydrolysed 12% w/v reconstituted skimmed milk was fermented with L. helveticus 881315 and Flavourzyme®, as detailed below.

5 L of 12% w/v RSM medium from Example 1 was inoculated with an aliquot of L. helveticus 881315 culture of Example 2 so as to obtain a 1% v/v bacterial culture, and with Flavourzyme®(Flavourzyme®1000 L (EC 3.4.11.1, an aminopeptidase with an activity of 1000 leucine aminopeptidase (LAPU/g), Novozymes Australia, North Rocks, NSW, Australia) (0.1% w/w). The resulting culture was incubated at 37° C. for 8-12 h with agitation (200-225 rpm). The incubation was conducted in a jacketed thermostatic water bath bioreactor (5 L capacity; Bio-Stat A plus) held at a constant temperature. The medium was continuously stirred by stirrer blades (200-225 rpm). The pH during fermentation was measured using a sterile pH electrode (DPAS Ingold, Paris, France) connected to a transmitter (Demca 3B 1015; Thermo Fisher Scientific, Alfortville, France). The pH electrode was calibrated before inoculating the medium. pH was measured to be 6.5 at time zero, and pH 3.4 at 8-12 h of fermentation. After the fermentation process, the fermented liquid product was heat-treated at 85° C. for 15 min to kill bacteria and inactivate the enzyme.

The fermented liquid product was cooled to room temperature then centrifuged at about 4000 × g (Sorvall RT7, Newtown, CT USA) at 4° C. for 30 min. The supernatant was freeze-dried for 72 h (Freeze-drier model FD-300; Air vac Engineering Pty. Ltd., VIC Australia) to obtain a powder product comprising the peptide composition. The powder could be stored at 4-5° C.

### Example 4: Fermentation of Pre-Treated Milk Composition Substrate WithL helveticus Strains 881315 and 881188, and Flavourzyme®

For producing antihypertensive peptide compositions, pasteurized and mechanically hydrolysed 12% w/v reconstituted skimmed milk was fermented with L. helveticus strains 881315 and 881188, and Flavourzyme®, as detailed below.

5 L of 12 w/v RSM medium from Example 1 was inoculated with an aliquot of each of L. helveticus 881315 culture and L. helveticus 881315 culture of Example 2 so as to obtain a 1% v/v bacterial culture of each strain (total bacterial culture 2% v/v), and with Flavourzyme®(0.1% w/w). The resulting culture was incubated at 37° C. for 8-12 h with agitation (200-225 rpm). The incubation was conducted in a jacketed thermostatic water bath bioreactor (5 L capacity; Bio-Stat A plus) held at a constant temperature. The medium was continuously stirred by stirrer blades (200-225 rpm). The pH during fermentation was measured using a sterile pH electrode (DPAS Ingold, Paris, France) connected to a transmitter (Demca 3B 1015; Thermo Fisher Scientific, Alfortville, France). The pH electrode was calibrated before inoculating the medium. pH was measured to be 6.5 at time zero and pH 3.4 at 8-12 h of fermentation. After the fermentation process, the fermented liquid product was heat-treated at 85° C. for 15 min to kill bacteria and inactivate the enzyme.

The fermented liquid product was cooled to room temperature then centrifuged at 4000 × g at 4° C. for 30 min. The supernatant was freeze-dried for 72 h to obtain a powder product comprising the peptide composition. The powder could be stored at 4-5° C.

### Example 5: Fermentation of Pre-Treated Milk Composition Substrate With L helveticus 881315 and Flavourzyme®, Followed by Further Fermentation With K. marxianus

Peptide compositions obtained by fermentation with L. helveticus 881315 and Flavourzyme®can be further fermented using K. marxianus.

The fermented liquid product of Example 3 was cooled to 37° C. then treated with a further amount of 12% w/v RSM medium of Example 1 (10% v/v), and the resulting mixture was inoculated with K. marxianus LAF4 of Example 2 so as to obtain a 1% v/v yeast culture. The resulting culture was incubated at 37° C. for 8 h with agitation (200-225 rpm). The incubation was conducted in a jacketed thermostatic water bath bioreactor (5 L capacity; Bio-Stat A plus) held at a constant temperature. The medium was continuously stirred by stirrer blades (200-225 rpm). After the fermentation process, the fermented liquid product was heat-treated at 85° C. for 15 min to inactivate the yeast.

The fermented liquid product was cooled to room temperature then centrifuged at 4000 × g at 4° C. for 30 min. The supernatant was freeze-dried for 72 h (Freeze-drier model FD-300; Air vac Engineering Pty. Ltd., VIC Australia) to obtain a powder product comprising the peptide composition.

### Example 6: Fermentation of Pre-Treated Milk Composition Substrate With L helveticus Strains 881315 and 881188 and Flavourzyme®, Followed by Further Fermentation With K. marxianus

Peptide compositions obtained by fermentation with L. helveticus strains 881315 and 881188 and Flavourzyme®can be optionally further fermented using K. marxianus.

The fermented liquid product of Example 4 was cooled to 37° C. then treated with a further amount of 12% w/v RSM medium of Example 1 (10% v/v), and the resulting mixture was inoculated with K. marxianus LAF4 of Example 2 so as to obtain a 1% v/v yeast culture. The resulting culture was incubated at 37° C. for 8 h with agitation (200-225 rpm). The incubation was conducted in a jacketed thermostatic water bath bioreactor (5 L capacity; Bio-Stat A plus) held at a constant temperature. The medium was continuously stirred by stirrer blades (200-225 rpm). After the fermentation process, the fermented liquid product was heat-treated at 85° C. for 15 min to inactivate the yeast.

The fermented liquid product was cooled to room temperature then centrifuged at 4000 × g at 4° C. for 30 min. The supernatant was freeze-dried for 72 h (Freeze-drier model FD-300; Air vac Engineering Pty. Ltd., VIC Australia) to obtain a powder product comprising the peptide composition.

### Example 7: ACE-Inhibitory Activity of the Fermented Peptide Compositions

Angiotensin-converting enzyme inhibitory activity of the peptide compositions produced as described above was measured.

ACE-inhibitory activity was determined according to a previously described method (Donkor et al., 2007). Briefly, the freeze-dried powder obtained in Examples 4 and 6 (40 mg) was dissolved in 2 mL Tris buffer (50 mM, pH 8.3) containing 300 mM sodium chloride. ACE enzyme and hippuryl-L-histidyl-L-leucine (HHL) (Sigma, St. Louis, MO USA) were prepared in Tris buffer. Fifty µL of 3.0 mM HHL, 50 µL of 1.25 MU ACE enzyme from rabbit lung (Sigma, Sigma-Aldrich, Castle Hill NSW, Australia), and 50 µL of experimental samples were placed in a glass tube and incubated for 1 h at 37° C. ensuring mixing for the first 30 min. Glacial acetic acid (150 µL) was added to stop the reaction. The reaction mixture was stored at - 20° C. before further analysis of released hippuric acid (HA) by HPLC. An external standard curve of hippuric acid was prepared to quantify the resultant hippuric acid in fermented samples. An aliquot (20 µL) of the mixture was injected into Gemini® C18 110 Å (100 mm x 4.6 mm, 5 µm) column (Phenomenex, Pty Ltd., NSW Australia) using Varian HPLC equipped with an auto sampler. The separation was conducted at room temperature (~22° C.) at a mobile phase flow rate of 0.6 mL min-1. The mobile phase consisting of 12.5 % (v/v) acetonitrile (Merck Pty. Ltd., VIC Australia) in MilliQ-water, and pH was adjusted to 3.0 using glacial acetic acid. Ultraviolet-visible detector was set at 228 nm.

The % ACE-I was calculated as follows:

$$A C E I \% = \frac{H A \left(c o n t r o l\right) - H A \left(s a m p l e\right)}{H A \left(c o n t r o l\right)} \times 100$$

Where ACE-I = angiotensin converting enzyme inhibition.

The ACE-inhibitory activity of peptide composition obtained in Example 4 was measured to be 95.6 %, and that of Example 6 was measured to be 100 %.

### Example 8: Effect of the Fermented Peptide Compositions on Body Weight and Food Intake

Effect of the fermented peptide compositions on body weight and food intake was measured in spontaneously hypertensive rats (SHR).

Fourteen-week-old male SHR were fed for ten weeks with either a standard chow (NC), the fermented peptide compositions added to chow (FC), or skim milk powder added to chow (NFC). Food intake and body weight were measured daily.

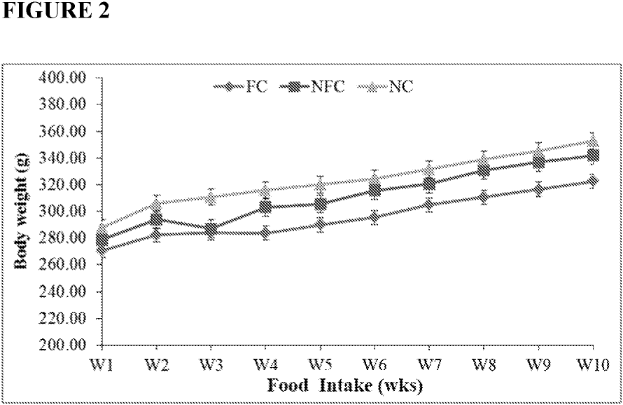

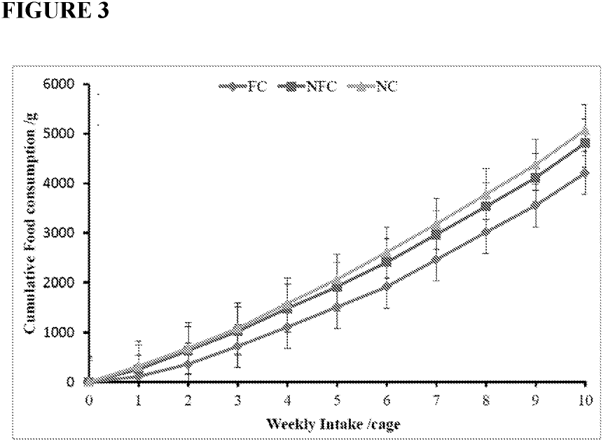

Body weight of SHR subjects was first recorded upon arrival (weight, 250 ± 5 g). All three groups (NC, FC and NFC) significantly increased body weight (FIG. 2) and food intake (FIG. 3) during the last seven weeks. However, FIG. 2 shows that lower body weight was observed for the FC group compared with the control groups fed with wither either the standard chow (NC) or skim milk powder added to chow (NFC). At the same time, the calculated total energy intake showed no significant differences between the FC, NFC and NC chow compositions (259.27, 212.77 and 238.14 MJ, respectively) (P < 0.05), as shown in Table 1.

Previous work had demonstrated that the decrease body fat in ACE-KO mice is independent of food intake and appears to be due to a high energy expenditure related to increased metabolism of fatty acids in the liver, with the additional effect of increased glucose tolerance. In the current study, it was found that despite similar food energy consumption in all three groups, the FC group weighed less at the end of the study than the NC and NFC groups (FIG. 2). There were no significant differences between the weights of organs of three different treatment groups related to the body weight. The increase in energy expenditure was independent of locomotor activity and appears to be mediated by increased fatty acid oxidation in the liver, so it is considered that the differences in body fat and energy expenditure could be due to differences in fat metabolism.

TABLE 1

Composition of the feeds used in this study.
Addition rate (g/100g)

| Ingredients | Control feed (standard rat chow, NC) | Skim milk powder control-containing chow feed (NFC) w | Fermented peptide composition-containing feed (FC) |
|---|---|---|---|
| Sucrose | 10.00 | 10.00 | 10.00 |
| Freeze dried fermented SM containing Peptides (FSMP) | 0.00 | 0.00 | 44.48 |
| Skim milk powder (SMPOC) | 0.00 | 44.48 | 0.00 |
| Canola oil | 4.00 | 3.50 | 3.50 |
| Cellulose | 5.00 | 5.00 | 5.00 |
| Starch | 19.26 | 19.26 | 19.16 |
| Dextrinised Starch | 15.50 | 15.50 | 15.50 |
| DL-methionine | 0.18 | 0.18 | 0.18 |
| AIN-93-trace minerals | 0.14 | 0.14 | 0.14 |
| Calcium carbonate | 0.13 | 0.06 | 0.06 |
| Sodium chloride | 0.26 | 0.18 | 0.18 |
| Potassium sulphate | 0.45 | 0.45 | 0.45 |
| AIN-93-Vitamins | 1.00 | 1.00 | 1.00 |
| Choline chloride 75% W/W | 0.25 | 0.25 | 0.25 |
| Blue food colour (10%) | 0.00 | 0.00 | 0.10 |
| Energy content (MJ)* | 15.1±0.012 | 18.4±0.002 | 16.9±0.011 |
| Total Energy intake /each group (MJ) | 212.77±0.004 | 259.27±0.001 | 238.14±0.002 |

*this represents the calorimetric value of the sample.

In conclusion, both food intake and body weight were affected by the fermented peptide compositions. The results showed that there was a significant reduction in body weight. It is believed that the observed effect may be a result of increased metabolic rate due to inhibition of angiotensin converting enzyme activity by the fermented peptide compositions.

### Example 9: Beverages Comprising the Antihypertensive Peptide Composition

The peptide compositions of the present disclosure can be used to produce beverages such as a fermented milk drink.

Powder products as prepared in Examples 4 and 6 were reconstituted with drinking quality water, then treated with blueberry food flavouring (2-6% w/v) and a non-caloric sweetener (5% w/v) to produce a flavoured fermented milk drink.

### Example 10: Food Products Comprising the Antihypertensive Peptide Composition

The hydrolysed peptide compositions of the present disclosure can be used to produce food supplements or food products.

Powder products as prepared in Examples 3 to 6 can be used as such or mixed with food additives to produce food supplements. Alternatively, the powder can be mixed with other food additives and food ingredients to produce food products comprising the peptide compositions, such as yogurt or spreads.

### Example 11: Pharmaceutical Products Comprising the Antihypertensive Peptide Composition

The peptide compositions of the present disclosure can be used to produce pharmaceutical compositions.

Powder products as prepared in Examples 3 to 6 can be processed to fill oral unit dosage forms (e.g. hard shell gelatin capsules), or compressed into tablets for oral delivery of the hydrolysed peptidic compositions. Alternatively, the powder can be mixed with pharmaceutically-acceptable excipients prior to production of such oral unit dosage forms.

### REFERENCES

Alhaj, O. A., Kanekanian, A. D., & Peters, A. C. (2007). Investigation on whey proteins profile of commercially available milk-based probiotics health drinks using fast protein liquid chromatography (FPLC). British Food Journal, 109(6), 469-480.

Boustany, C. M., Bharadwaj, K., Daugherty, A., Brown, D. R., Randall, D. C., & Cassis, L. A. (2004). Activation of the systemic and adipose renin-angiotensin system in rats with diet-induced obesity and hypertension. American Journal of Physiology -Regulatory, Integrative and Comparative Physiology, 287(4), R943-R949. doi: 10.1152/ajpregu.00265.2004

Cameron, A. J., Welborn, T. A., Zimmet, P. Z., Dunstan, D. W., Owen, N., Salmon, J., Shaw, J. E. (2003). Overweight and obesity in Australia: the 1999-2000 Australian diabetes, obesity and lifestyle study (AusDiab). Medical Journal of Australia, 178(9), 427-432.

Contor, L. (2001). Functional food science in europe. Nutrition, metabolism, and cardiovascular diseases: NMCD, 11(4 Suppl), 20-23.

Donkor, O., Henriksson, A., Singh, T. K., Vasiljevic, T., & Shah, N. P. (2007). ACE inhibitory activity of probiotic yoghurt. International Dairy Journal, 17(11), 1321-1331. doi: 10.1016/j.idairyj.2007.02.009.

Donkor, O., Henriksson, A., Vasiljevic, T., & Shah. (2007). Proteolytic activity of dairy lactic acid bacteria and probiotics as determinant of growth and in vitro angiotensin-converting enzyme inhibitory activity in fermented milk. Lait, 87(1), 21-38.

Donkor, O., Henriksson, A., Vasiljevic, T., & Shah, N. P. (2006). Effect of acidification on the activity of probiotics in yoghurt during cold storage. International Dairy Journal, 16(10), 1181-1189. doi: 10.1016/j.idairyj.2005.10.008.

Donkor, O., Henriksson, A., Vasiljevic, T., & Shah, N. P. (2005). Probiotic strains as starter cultures improve angiotensin-converting enzyme inhibitory activity in soy yogurt. Journal of Food Science, 70(8), m375-m381. doi: 10.1111/j.1365-2621.2005.tb11522.x.

Erdmann, K., Cheung, B. W., & Schroder, H. (2008). The possible roles of food-derived bioactive peptides in reducing the risk of cardiovascular disease. The Journal of Nutritional Biochemistry, 19(10), 643-654.

FitzGerald, C., Gallagher, E., Tasdemir, D., & Hayes, M. (2011). Heart health peptides from macroalgae and their potential use in functional foods. Journal of Agricultural and Food Chemistry, 59(13), 6829-6836.

FitzGerald, R. J., & Murray, B. A. (2006). Bioactive peptides and lactic fermentations. International Journal of Dairy Technology, 59(2), 118-125. doi: 10.1111/j.1471-0307.2006.00250.

FitzGerald, R., Murray, B., & Walsh, D. (2004). Hypotensive peptides from milk proteins. The Journal of Nutrition, 134(4), 980-988.

FitzGerald, R. J., & Meisel, H. (2003). Milk protein hydrolysates and bioactive peptides. In P. F. Fox & P. L. H. McSweeney (Eds.), Advanced Dairy Chemistry-1 Proteins (pp. 675-698): Springer US.

FitzGerald, R., & Meisel, H. (2000). Milk protein-derived peptide inhibitors of angiotensin-I-converting enzyme. British Journal of Nutrition, 84, 33-37. 5.

FitzGerald, R. J. (1998). Potential Uses of Caseinophosphopeptides. International Dairy Journal, 8(5-6), 451-457. doi: http://dx.doi.org/10.1016/50958-6946(98)00068.

Gobbetti, M., Minervini, F., & Rizzello, C. G. (2004). Angiotensin I-converting enzyme-inhibitory and antimicrobial bioactive peptides. International Journal of Dairy Technology, 57(2-3), 173-188.

Gobbetti, M., Stepaniak, L., De Angelis, M., Corsetti, A., & Di Cagno, R. (2002). Latent bioactive peptides in milk proteins: Proteolytic activation and significance in dairy processing. Critical Reviews in Food Science and Nutrition, 42(3), 223-239.

Gobbetti, M., Ferranti, P., Smacchi, E., Goffredi, F., & Addeo, F. (2000). Production of Angiotensin-I-Converting-enzyme-inhibitory peptides in fermented milks started by Lactobacillus delbrueckii subsp. bulgaricus SSl and Lactococcus lactis subsp. cremoris

FT4. Applied and Environmental Microbiology, 66(9), 3898- 3904. doi: 10.1128/aem.66.9.3898-3904.2000.

Guo, S., Goetze, J. P., Jeppesen, J. L., Burnett, J. C., Olesen, J., Jansen-Olesen, I., & Ashina, M. (2015). Effect of natriuretic peptides on cerebral artery blood flow in healthy volunteers.Peptides,74,33-42.doi:http://dx.doi.org/10.1016/j. peptides. 2015.09.008.

Hall, J., Brands, M., Hildebrandt, D., Kuo, J., & Fitzgerald, S. (2000). Role of sympathetic nervous system and neuropeptides in obesity hypertension. Brazilian Journal of Medical and Biological Research, 33(6), 605-618.

Hekmat, S., Soltani, H., & Reid, G. (2009). Growth and survival of Lactobacillus reuteri RC-14 and Lactobacillus rhamnosus GR-1 in yogurt for use as a functional food. Innovative food science & emerging technologies, 10(2), 293-296.

Kawase, M., Hashimoto, H., Hosoda, M., Morita, H., & Hosono, A. (2000). Effect of administration of fermented milk containing Whey protein concentrate to rats and healthy men on serum lipids and blood pressure. Journal of Dairy Science, 83(2), 255-263.

Khavinson V., Linkova N., Dyatlova A., Kuznik B., Umnov R. (2020). Peptides: Prospects for Use in the Treatment of COVID-19. Molecules, 25(19), 4389.

Kitts, D. D., & Weiler, K. (2003). Bioactive proteins and peptides from food sources. Applications of bioprocesses used in isolation and recovery. Current pharmaceutical design, 9(16), 1309-1323.

López-Fandiño, R., Otte, J., & van Camp, J. (2006). Physiological, chemical and technological aspects of milk-protein-derived peptides with antihypertensive and ACE-inhibitory activity. International Dairy Journal, 16(11), 1277-1293. doi: http://dx.doi.org/10.1016/j.idairyj.2006.06.004.

Marinik, E. L., Frisard, M. I., Hulver, M. W., Davy, B. M., Rivero, J. M., Savla, J. S., & Davy, K. P. (2013). Angiotensin ii receptor blockade and insulin sensitivity in overweight and obese adults with elevated blood pressure. Therapeutic Advances in Cardiovascular Disease, 7(1), 11-20. doi: 10.1177/1753944712471740

Marteau, P., Flourie, B., Pochart, P., Chastang, C., Desjeux, J.-F., & Rambaud, J.-C. (1990). Effect of the microbial lactase (EC 3.2. 1.23) activity in yoghurt on the intestinal absorbance of lactose: an in vivo study in lactase-deficient humans. British Journal of Nutrition, 64(01), 71-79.

Matsoukas J., Apostolopoulos V., Zulli A., Moore G., Kelaidonis K., Moschovou K., Mavromoustakos T. (2021). From Angiotensin II to Cyclic Peptides and Angiotensin Receptor Blockers (ARBs): Perspectives of ARBs in COVID-19 Therapy. Molecules, 26(3), 618.

Meisel, H. (1998). Overview on Milk Protein-derived Peptides. International Dairy Journal, 8(5-6), 363-373. doi: http: //dx.doi. org/10.1016/S0958-6946(98) 00059-4.

Ozer, B., Kirmaci, H. A., Oztekin, S., Hayaloglu, A., & Atamer, M. (2007). Incorporation of microbial transglutaminase into non-fat yogurt production. International Dairy Journal, 17(3), 199-207.

Rajapakse, N., Mendis, E., Byun, H.-G., & Kim, S.-K. (2005). Purification and in vitro antioxidative effects of giant squid muscle peptides on free radical-mediated oxidative systems. The Journal of Nutritional Biochemistry, 16(9), 562-569. doi: http://dx.doi.org/10.1016/j.jnutbio.2005.02.005.

Roberfroid, M. B. (2000). Prebiotics and probiotics: are they functional foods? The American Journal of Clinical Nutrition, 71(6), 1682s-1687.

Roberfroid, M. B. (1999). Concepts in functional foods: the case of inulin and oligofructose. The Journal of Nutrition, 129(7), 1398S-1401.

Santos, A., San Mauro, M., & Diaz, D. M. (2006). Prebiotics and their long-term influence on the microbial populations of the mouse bowel. Food Microbiology, 23(5), 498-503.

Seppo, L., Jauhiainen, T., Poussa, T., & Korpela, R. (2003). A fermented milk high in bioactive peptides has a blood pressure-lowering effect in hypertensive subjects. American Journal of Clinical Nutrition, 77(2), 326-330.

Shah, N. P. (2007). Functional cultures and health benefits. International Dairy Journal, 17(11), 1262-1277. doi: http://dx.doi.org/10.1016/j.idairyj.2007.01.014.

Stanton, C., Ross, R. P., FitzGerald, G. F., & Van Sinderen, D. (2005). Fermented functional foods based on probiotics and their biogenic metabolites. Current Opinion in Biotechnology, 16(2), 198-203.

Silva, S. V., & Malcata, F. X. (2005). Caseins as source of bioactive peptides. International Dairy Journal, 15(1),1-15. doi:http://dx.doi.org/10.1016/j. idairyj.2004.04.009.

Tuohy, K. M., Probert, H. M., Smejkal, C. W., & Gibson, G. R. (2003). Using probiotics and prebiotics to improve gut health. Drug discovery today, 8(15), 692-700.

Unal, G., & Akalin, A. S. (2012). Antioxidant and angiotensin-converting enzyme inhibitory activity of yoghurt fortified with sodium calcium caseinate or whey protein concentrate. Dairy Science and Technology, 92(6), 627-639.

Van der Burg-Koorevaar, M. C., & Schalk, J. (2010). Hydrolysed casein product comprising tripeptides IPP and/or VPP: Google Patents.

Williams, C. M., & Jackson, K. G. (2002). Inulin and oligofructose: effects on lipid metabolism from human studies. British Journal of Nutrition, 87(S2), S261-S264.

World Health Organization. (2013). A global brief on hypertension: Silent killer, global public health crisis. Cardiovascular disease. Retrieved 2 February, 2013, from http://www.who.int/cardiovascular_diseases/publications/global_brief_hyperten sion/en/.

World Health Organization. (2003). diet, nutrition and the prevention of chronic diseases. WHO Library Cataloguing-in-Publication Data. (WHO technical report series ; 916), from http://whqlibdoc.who.int/trs/who_trs_916.pdf.

Food and Agriculture Organization of the United Nations, & World Health Organization. (2002). Guidelines for the evaluation of probiotics in food: Report of a joint FAO/WHO working group on drafting guidelines for the evaluation of probiotics in food. Accessed 2 February, 2013, from http://www.fda.gov/ohrms/dockets/dockets/95s0316/95s-0316-rpt0282-tab-03-ref-19-joint-faowho-vo1219.pdf.

Yamamoto, N., Mine, Y., Li-Chan, E., & Jiang, B. (2010). Functional food products with antihypertensive effects. Bioactive Proteins and Peptides as Functional Foods and Nutraceuticals, 29, 169.

1. A process for producing an antihypertensive peptide composition, comprising:
a) fermenting a milk composition with a Lactobacillus helveticus and a peptidase selected from the group consisting of an aminopeptidase and a serine endopeptidase, thereby at least partially hydrolysing peptides present in the composition; and
b) heat-treating the product of step a) so as to inactivate the Lactobacillus helveticus and the peptidase;
c) admixing the at least partially hydrolysed peptides with a further quantity of milk composition and with Kluyveromyces marxianus, and fermenting the mixture to produce the antihypertensive peptide composition.

2. A process as claimed in claim 1, wherein the milk composition is reconstituted skimmed milk.

3. A process as claimed in claim 1 or claim 2, wherein the milk composition is pasteurised.

4. A process as claimed in claim 3, wherein the milk composition is pasteurised by heating it at a temperature in the range of from 85° C. to 95° C. for a period in the range of from 15 minutes to 18 minutes.

5. A process as claimed in any of claims 1 to 4, wherein, prior to step a), the milk composition is subjected to mechanical agitation.

6. A process as claimed in claim 5, wherein mechanical agitation is carried by agitating the milk composition at a rate in the range of from 200 to 225 revolutions per minute, at a temperature of about 37° C., and for a period in the range of from 30 minutes to 1 hour.

7. A process as claimed in any of claims 1 to 6, wherein, prior to step a), the Lactobacillus helveticus is activated by carrying out three iterations of incubating in MRS medium at about 37° C. for about 18 hours.

8. A process as claimed in any of claims 1 to 7, wherein the peptidase is an aminopeptidase.

9. A process as claimed in claim 8, wherein the aminopeptidase is present in a composition having an activity of about 1000 leucine amino-peptidase (LAPU g⁻¹).

10. A process as claimed in any of claims 1 to 7, wherein the peptidase is a serine endopeptidase.

11. A process as claimed in claim 10, wherein the serine endopeptidase is present in a composition having an activity of at least 2.4 Anson units per g.

12. A process as claimed in any of claims 1 to 11, wherein, to commence fermentation, an amount in the range of from 1 to 2% v/v of a composition comprising the Lactobacillus helveticus, and an amount in the range of from 0.04 to 0.45% w/w of a peptidase composition is added to the milk composition.

13. A process as claimed in any of claims 1 to 12, wherein the first fermentation step is carried out for a period in the range of from 8 to 12 hours.

14. A process as claimed in any of claims 1 to 13, wherein in step b) the composition is subjected to a temperature in the range of from 80° C. to 90° C. for a period in the range of from 12 to 15 minutes.

15. A process as claimed in any of claims 1 to 14, wherein the further quantity of milk composition is pasteurised reconstituted skimmed milk.

16. A process as claimed in any of claims 1 to 15, wherein the second fermentation step is carried out for a period in the range of from 8 to 12 hours.

17. A process as claimed in any of claims 1 to 16, wherein, following fermentation of the mixture containing Kluyveromyces marxianus, the composition is subjected to heat treatment to inactivate the Kluyveromyces marxianus.

18. A process as claimed in claim 17, wherein, following fermentation of the mixture containing Kluyveromyces marxianus, the composition is subjected to a temperature in the range of from 80° C. to 90° C. for a period in the range of from 6 to 30 minutes.

19. A process as claimed in any of claims 1 to 18, wherein, following heat treatment, antihypertensive peptide is separated from Kluyveromyces marxianus.

20. A process as claimed in claim 19, wherein, following separation from Kluyveromyces marxianus, the antihypertensive peptide composition is lyophilised.

21. A process for producing an antihypertensive peptide composition, comprising:
i) subjecting a milk composition to mechanical agitation;
ii) fermenting the product of step i) with a Lactobacillus helveticus and a peptidase selected from the group consisting of an aminopeptidase and a serine endopeptidase, thereby at least partially hydrolysing peptides present in the composition; and
iii) heat-treating the product of step ii) so as to inactivate the Lactobacillus helveticus and the peptidase.

22. A process as claimed in claim 21, wherein the milk composition is reconstituted skimmed milk.

23. A process as claimed in claim 21 or claim 22, wherein the milk composition is pasteurised.

24. A process as claimed in claim 23, wherein the milk composition is pasteurised by heating it at a temperature in the range of from 85° C. to 95° C. for a period in the range of from 15 minutes to 18 minutes.

25. A process as claimed in any of claims 21 to 24, wherein in step i) mechanical agitation is carried by agitating the milk composition at a rate in the range of from 200 to 225 revolutions per minute, at a temperature of about 37° C., and for a period in the range of from 30 minutes to 1 hour.

26. A process as claimed in any of claims 21 to 25, wherein, prior to step ii), the Lactobacillus helveticus is activated by carrying out three iterations of incubating in MRS medium at about 37° C. for about 18 hours.

27. A process as claimed in any of claims 21 to 26, wherein the peptidase is an aminopeptidase.

28. A process as claimed in claim 27, wherein the aminopeptidase is present in a composition having an activity of about 1000 leucine amino-peptidase (LAPU g⁻¹).

29. A process as claimed in any of claims 21 to 26, wherein the peptidase is a serine endopeptidase.

30. A process as claimed in claim 29, wherein the serine endopeptidase is present in a composition having an activity of at least 2.4 Anson units per g.

31. A process as claimed in any of claims 21 to 30, wherein, to commence fermentation, an amount in the range of from 1 to 2% v/v of a composition comprising the Lactobacillus helveticus, and an amount in the range of from 0.04 to 0.45% w/w of a peptidase composition is added to the milk composition.

32. A process as claimed in any of claims 21 to 31, wherein step ii) is carried out for a period in the range of from 8 to 12 hours.

33. A process as claimed in any of claims 21 to 32, wherein in step iii) the composition is subjected to a temperature in the range of from 80° C. to 90° C. for a period in the range of from 12 to 15 minutes.

34. A process as claimed in any of claims 21 to 33, wherein, following step iii), antihypertensive peptides are separated from Lactobacillus helveticus.

35. A process as claimed in claim 34, wherein, following separation from Lactobacillus helveticus, the antihypertensive peptide composition is lyophilised.

36. A process as claimed in any of claims 21 to 35, wherein following step iii) the antihypertensive peptide composition is admixed with a further quantity of milk composition and with Kluyveromyces marxianus, and the resulting mixture is fermented to produce a further antihypertensive peptide composition.

37. A process as claimed in claim 36, wherein the further quantity of milk composition is pasteurised reconstituted skimmed milk.

38. A process as claimed in claim 37, wherein the pasteurised reconstituted skimmed milk has been subjected to mechanical agitation.

39. A process as claimed in any of claims 36 to 38, wherein the mixture containing Kluyveromyces marxianus is fermented for a period in the range of from 8 to 12 hours.

40. A process as claimed in any of claims 36 to 39, wherein, following fermentation of the mixture containing Kluyveromyces marxianus, the composition is subj ected to heat treatment at a temperature in the range of from 80° C. to 90° C. for a period in the range of from 6 to 30 minutes, so as to inactivate the Kluyveromyces marxianus.

41. A process as claimed in any of claims 36 to 40, wherein, following heat treatment, antihypertensive peptide is separated from Kluyveromyces marxianus.

42. A process as claimed in claim 41, wherein, following separation from Kluyveromyces marxianus, the antihypertensive peptide composition is lyophilised.

43. A process as claimed in any of claims 1 to 42, wherein the peptide composition has antioxidant activity, antibacterial activity, anticancer activity, antihypertensive activity and/or facilitates weight loss.

44. An antihypertensive peptide composition produced according to any of claims 1 to 43.

45. A dosage form comprising the antihypertensive peptide composition of claim 44.

46. A dosage form as claimed in claim 45, which is in the form of a liquid, powder, tablet or capsule.

47. A food product or supplement comprising the antihypertensive peptide composition of claim 44.

48. A food product as claimed in claim 47, wherein the food product is a spread or a yogurt.

49. A powdered milk formula comprising the antihypertensive peptide composition of claim 44.

50. A beverage comprising the antihypertensive peptide composition of claim 44.

51. A beverage as claimed in claim 50, wherein the beverage is a milk drink.

52. A dosage form, food product, food supplement, powdered milk formula or beverage as claimed in any of claims 45 to 51, which comprises one or more of a flavouring, antioxidant and sweetener.

53. A method of treating, preventing, or reducing the likelihood of a disease or disorder in a subject, comprising administering an effective amount of an antihypertensive peptide composition, dosage form, food product, food supplement, powdered milk formula or beverage as claimed in any of claims 44 to 52 to the subject.

54. A method as claimed in claim 53, wherein the disease or disorder is hypertension, a cardiovascular disease or disorder, heart disease, an immune system disease or disorder, a bacterial infection, a viral infection, cancer, being overweight, or obesity.

55. A method as claimed in claim 54, wherein the disease or disorder is selected from the group consisting of hypertension and heart disease.

56. A method as claimed in claim 54, wherein the disease or disorder is being overweight or obesity.

57. Use of an antihypertensive peptide composition, dosage form, food product, food supplement, powdered milk formula or beverage as claimed in any of claims 44 to 52, for the manufacture of a medicament for the treatment, prevention, or reducing the likelihood of a disease or disorder selected from the group consisting of hypertension, a cardiovascular disease or disorder, heart disease, an immune system disease or disorder, a bacterial infection, a viral infection, cancer, being overweight, and obesity.

58. An antihypertensive peptide composition, dosage form, food product, powdered milk formula or beverage as claimed in any of claims 44 to 52, for use in the treatment or prevention of, or for use in reducing the likelihood of, a disease or disorder.

59. An antihypertensive peptide composition, dosage form, food product, food supplement, powdered milk formula or beverage for use as claimed in claim 58, wherein the disease or disorder is selected from the group consisting of hypertension, a cardiovascular disease or disorder, heart disease, an immune system disease or disorder, a bacterial infection, a viral infection, cancer, being overweight, and obesity.
